(19) United States
(12) Patent Application Publication
(10) Pub. No.: US 2017/0231910 A1
(43) Pub. Date: Aug. 17, 2017

(54) PHARMACEUTICAL FORMULATIONS OF CHELATING AGENTS AS A METAL REMOVAL TREATMENT SYSTEM

(71) Applicants: Mark E. HAYES, San Francisco, CA (US); Charles O. NOBLE, San Francisco, CA (US); Francis C. SZOKA, JR., San Francisco, CA (US); ZONEONE PHARMA, INC., San Francisco, CA (US)

(72) Inventors: Mark E. HAYES, San Francisco, CA (US); Charles O. NOBLE, San Francisco, CA (US); Francis C. SZOKA, JR., San Francisco, CA (US)

(21) Appl. No.: 15/503,001

(22) PCT Filed: Aug. 12, 2015
(86) PCT No.: PCT/US15/44878

§ 371 Date: Feb. 9, 2017

Related U.S. Application Data
(60) Provisional application No. 62/037,044, filed on Aug. 13, 2014.

(51) Int. Cl. A61K 9/127; B01J 13/08; B01J 13/20; A61K 31/16; A61K 31/4196

(52) U.S. Cl. CPC A61K 9/127; A61K 9/1272; A61K 31/16; A61K 31/4196; B01J 13/203; B01J 13/08

(57) ABSTRACT

The present invention provides liposomes loaded with chelating agents, pharmaceutical formulations including these liposomes and methods of making chelating agent liposomes. Because the chelating agents are loaded in the liposome with high efficiencies, the liposomes are of use in treatment of metal ion overload in subjects. The liposomes can also contain essential trace metals to compensate for the off target effect of removal of endogenous non-target trace metals by administration of the chelator. The liposomes can include two or more different chelating agents of different structures and affinities for metal ions.

### CROSS-REFERENCES TO RELATED APPLICATIONS

This application claims the benefit under 35 U.S.C. §119(e) of U.S. Provisional Application No. 62/037,044 filed on Aug. 13, 2014, which is incorporated herein by reference in its entirety for all purposes.

### BACKGROUND OF THE INVENTION

This invention relates to the fields of pharmaceutical formulation, methods for efficiently making them and the uses of the resulting compositions in metal chelation therapy. The formulations include a mixture consisting of one or more chelating agents on the outside of a lipid vesicle with one or more chelating agents encapsulated with a suitable multivalent salt in the interior aqueous compartment of a lipid vesicle. The metal removal treatment system consisting of a mixture of lipid vesicle and the chelating agent(s) is formulated in a pharmaceutically acceptable diluent for administration into a patient.

### DESCRIPTION OF THE RELATED ART

The element iron is the most abundant metal in humans and is essential for life because of its key role in oxygen transport. Healthy adults possess between 3 to 5 g of iron. The bulk of this iron is required for oxygen transport and is bound to hemoglobin in the red blood cell, the muscle oxygen storage protein myoglobin, or is stored by ferritin, hemosiderin or transferrin to prevent accumulation of redox-active (free) iron in sensitive sites.

Normal humans absorb between 1-2 mg iron per day in the Fe (II) form through the intestine to compensate for the 1 to 2 mg daily body loss of iron. Total iron levels in the body are regulated mainly through absorption from the intestine and the erythropoietic activity of the bone marrow. In healthy individuals an equilibrium is maintained between the sites of iron absorption, storage and utilization. Remarkably, humans lack any effective means to excrete excess iron; this can have fatal consequences for patients that require chronic blood transfusions.

In the United States, 10,000 to 20,000 patients with sickling disorders receive repeated blood transfusions. An estimated 4000 to 5000 patients with myelo-dysplastic syndromes and other forms of acquired refractory anemia require red-cell transfusions. The number of patients with transfusion-dependent thalassemia in the United States is about 1000. However, globally, almost 100,000 patients with thalassemia syndromes undergo transfusions.

In patients with thalassemia who undergo transfusion from infancy, iron-induced liver disease and endocrine disorders develop during childhood and are almost inevitably followed in adolescence by death from iron-induced cardiomyopathy. In patients with sickle cell anemia, iron-induced complications develop later, eventually, liver disease with cirrhosis as well as cardiac and pancreatic iron deposition appears. (Weinberg E D, Miklossy J. Iron withholding: a defense against disease. J Alzheimers Dis. 2008 May; 13(4):451-63. PMID: 18487852; Fleming R E, Ponka P., Iron overload in human disease. N Engl J Med. 2012 Jan. 26; 366(4):348-59. doi: 10.1056/NEJMra1004967.).

β-thalassemia patients are transfused with approximately two units of blood per month. Since each unit of blood contains about 220 mg of iron, this transfusion regime results in an average daily iron intake of 15-22 mg/day, which is significantly in excess of the normal daily intake of 1 mg/day. Since there is no physiological mechanism to eliminate iron from the body it builds up in liver, spleen, heart and other organs. (FIG. 1). The reason for this is that at the end of their life span, transfused red cells are phagocytosed by reticuloendothelial macrophages in the liver, bone marrow, and spleen. Their hemoglobin is digested, and the iron is freed from heme and released into the cytosol. Early in the course of transfusion therapy, most of this additional iron can be stored within reticuloendothelial macrophages. (for a review see, Brittenham, G M, Iron-Chelating Therapy for transfusional Iron Overload, N Engl J Med 2011; 364:146-56. PMID: 21226580). Gradually, limits on the capacity of macrophages to retain iron result in the release of excess iron into plasma. Transferrin binds the released iron, with an increase in the plasma iron concentration and transferrin saturation. When transferrin saturates, hepatocytes are recruited to serve as storage sites for the excess iron. With continued transfusion, macrophages and hepatocytes can no longer retain all the excess iron.

Iron then enters plasma in amounts that exceed the transport capacity of circulating transferrin. As a consequence, non-transferrin-bound iron appears in the plasma. Non-transferrin-bound plasma iron enters hepatocytes, cardiomyocytes, anterior pituitary cells, and pancreatic beta-cells. Iron accumulation leads to the generation of reactive oxygen species, resulting in tissue damage. Thus for effective iron chelation therapy, iron must be removed from both the plasma, from inside of macrophages and from affected cells in other tissues. Current chelation therapies effectively remove iron from plasma but are less efficient at removing iron from within cells such as the reticuloendothelial cells in the liver, spleen and bone marrow. The pharmaceutical formulation described herein effectively removes iron from both the plasma and from the reticuloendothelial cells and in one embodiment can decrease the removal of other trace metals.

Three iron-chelating agents are approved for use: parenteral deferoxamine mesylate (Desferal®), oral deferasirox (Exjade®) and oral deferiprone (Ferriprox®) (FIG. 2). Deferoxamine (also known as desferroxamine, desferroxamine B, DFO-B, DFOA, DFB) is an iron-binding compound produced by the bacterium Streptomyces pilosus. It is very water-soluble but poorly absorbed after oral administration and is rapidly cleared; consequently, continuous subcutaneous or intravenous administration of deroxamine is necessary. To be effective deferoxamine must be infused 5 to 7 days per week, 10 hours per day at a dose of 20-50 mg/kg/day. A 50 kg patient receiving a high dose, would get 70 grams of deferoxamine per month. If it were a 100 percent efficient it would remove 7.0 grams of iron. This is equivalent to iron contained in 280 units of blood. However the patient receives only 2 units of blood per month. Thus, the current deferoxamine medicine is very inefficient for removing iron and extraordinarily burdensome for patients to take. Furthermore current chelation therapies remove other trace metals including zinc and copper which may have adverse effects for the patients (de Virgiliis et al., Archives of Disease in Childhood 63:250-255, 1988). The liposome chelating agent formulation described here can be given once or twice a month and requires only 10-15 grams of deferoxamine.

In contrast to deferoxamine, the synthetic chelating agent deferasirox has a very low water-solubility, is well absorbed from the gastro-intestinal tract, undergoes enterohepatic recycling and is cleared from the circulation slowly (Nick, Deferasirox (Exjade®, ICL670) Preclinical Overview, Seminars in Hematology 44:S12-S15, 2007). Deferasirox forms complexes with plasma iron, but deferasirox-iron complexes are eliminated predominantly through a hepatobiliary route. Hepatocytes more readily take up deferasirox, which chelates hepatocellular iron. The deferasirox-iron complexes are then excreted in the bile. Within cells, deferasirox chelates cytosolic iron, leading to ferritin degradation by the proteasome. The daily high dose of deferasirox is 40 mg/kg/day and the monthly dose for a 50 kg patient is 60 grams. If it were 100 percent efficient, deferasirox would chelate 4.5 grams of iron, about the amount of iron in about 20 units of blood. But deferasirox is not efficient in removing iron since a patient only receives 2 units of blood per month per month. In addition, the low solubility of defersirox makes it very difficult to encapsulate in liposomes.

Patients who take deferasirox run a higher risk of renal and hepatic failure. (Brittenham, G M, Iron-Chelating Therapy for transfusional Iron Overload, N Engl J Med 2011; 364:146-56. PMID: 21226580). Thus, the current treatment regimes for iron-overload disease are inefficient, lead to poor patient compliance and can result in life-threating complications. This invention provides a method to encapsulate high amounts of deferasirox in liposomes something that has not been accomplished in the past. Only 6.5 grams of liposome deferasirox need be delivered once a month to remove iron, so a lower amount of liposomes deferasirox can be administered less often. This would be safer and more convenient for the patient than the current way of administering deferasirox.

Other uses for chelating agents are in the removal of americium, arsenic, cadmium, copper, lead, plutonium, and uranium, from patients who have become exposed to these metals from environmental sources or radioisotope exposure disasters. Liposome chelating agents have a beneficial role to play in these situations where removal of metals from intracellular sites is required.

Lipid vesicles also known as liposomes are vesicle structures usually composed of a bilayer membrane of amphipathic molecules such as, phospholipids, entrapping an aqueous core. The diameters and morphology of various types of liposomes are illustrated in FIG. 2. Unilamellar liposomes with diameters less than 300 nm are best suited for administration via the parenteral route such as intravenously or subcutaneously. Chelating agents can either be encapsulated in the aqueous core or interdigitated in the bilayer membrane. Chelating agents that interdigitated in the membrane, transfer out of the liposome when it is diluted into the body. Importantly, chelating agents that are encapsulated in the aqueous core or held in complexes in the aqueous core are retained substantially longer than chelating agents in the bilayer. The use of liposomes with drugs encapsulated in the aqueous core for drug delivery and chelating agents for metal removal is well established (Rahman et al., 1973; Potsma et al., 1998; Drummond et al., 2008, review).

A variety of loading methods for encapsulating functional compounds in liposomes are available. Hydrophilic functional compounds, for example, can be encapsulated in liposomes by hydrating a mixture of the functional compounds and vesicle-forming lipids. This technique is called passive loading. The functional compound is encapsulated in the liposome as the nanoparticle is formed. The limitation to this method is that only a small fraction of the functional compound in the hydrating mixture is encapsulated into the liposome. This is because of the small internal volume of the liposome. For instance, a 100 nm diameter unilamellar liposome preparation created from 1 micromole of lipid encapsulates about 3 microliters of aqueous material. Thus a 100 umole lipid preparation can only passively encapsulate a theoretical maximum of about 30% of the starting dose and usually the encapsulated volume is much less than this, e.g., in the 10-20% range of the material initially in the rehydration medium. It is difficult to make liposome preparations with lipid amounts greater than 100 micromoles per mL because of the viscosity of the preparation.

The available lipid vesicle (liposome) production procedures for the encapsulation of water-soluble drugs can not overcome this limitation of the efficiency of the rehydration process (G. Gregoriadis, Liposome Technology: Liposome Preparation and Related Techniques, 3rd Edition (2006)). Thus manufacture of lipid vesicles that encapsulate sparingly water-soluble compounds (e.g., with a water solubility less than 2 mg/mL) in the aqueous inner compartment of the liposome or compounds with a molecular weight greater than 500 is difficult. This has caused the pharmaceutical industry to avoid liposomes to deliver sparingly water-soluble chelating agents or chelating agents with molecular weights greater than 500, for use in disease treatments in patients.

Hence, using passive loading, the final functional-compound-to-lipid ratio as well as the encapsulation efficiency are generally low. The concentration of drug in the liposome equals that of the surrounding fluid and drug not entrapped in the internal aqueous medium is washed away after the liposome is formed. The methods described in this invention overcome the current limitations to encapsulating chelating agents in liposomes.

The earliest publication dealing with the removal of metals from the body using chelating agents that can also bind to iron described the removal of plutonium from an animal with a liposome encapsulated diethylenetriaminepentaacetic acid (Rahman Y E, Rosenthal M W, Cerny E A. Intracellular plutonium removal by liposome-encapsulated chelating agent. Science (Wash. D.C.) 180:300-302, 1973). Following this work, a number of groups proposed that iron could be removed from the body by encapsulating deferoxamine in liposomes (Guilmette R A, Cerny E A, Rahman Y E. Pharmacokinetics of the iron chelating agent desferrioxamine as affected by liposome encapsulation: potential in treatment of chronic hemosiderosis. Life Sci. 22(4):313-2, 1978. PubMed PMID: 622008; Young S P, Baker E, Huehns E R., Liposome entrapped desferrioxamine and iron transporting ionophores: a new approach to iron chelation therapy. Br J Haematol. 41(3):357-63, 1979. PubMed PMID: 4633691:Lau E H, Cerny E A, Rahman Y E. Liposome-encapsulated desferrioxamine in experimental iron overload. Br J Haematol. 47(4):505-18, 1981. PubMed PMID: 7213574; Postma N S, Boerman O C, Oyen W J, Zuidema J, Storm G. Absorption and biodistribution of 111indium-labelled desferrioxamine (111In-DFO) after subcutaneous injection of 111In-DFO liposomes. J Control Release. 58(1):51-60, 1999 PubMed PMID: 10021489). However none of these prior publications enabled the treatment of patients with liposome encapsulated chelating agents for several reasons: the efficiency of the encapsulation process for an expensive chelating agent such as deferoxamine was too low so that too much chelating agent was lost in the process of making the liposomes, the amount of deferoxamine encapsulated in the liposome was not high enough (Guilmette R A, Cerny E A, Rahman Y E. Pharmacokinetics of the iron chelating agent desferrioxamine as affected by liposome encapsulation: potential in treatment of chronic hemosiderosis. Life Sci. 22(4):313-20, 1978 PubMed PMID: 622008; Young S P, Baker E, Huehns E R., Liposome entrapped desferrioxamine and iron transporting ionophores: a new approach to iron chelation therapy. Br J Haematol. 41(3):357-63, 1979 PubMed PMID: 4633691:Lau E H, Cerny E A, Rahman Y E. Liposome-encapsulated desferrioxamine in experimental iron overload. Br J Haematol. 47(4):505-18, 1981 PubMed PMID), so the amount of lipid that would have to be used to treat a patient was too high; or the diameter of the liposome used was too large to leave the injection site so the benefit of delivering the chelating agent into the liver was lost (Postma N S, Boerman O C, Oyen W J, Zuidema J, Storm G. Absorption and biodistribution of 111indium-labelled desferrioxamine (111In-DFO) after subcutaneous injection of 111In-DFO liposomes. J Control Release. 1999 Mar. 8; 58(1):51-60. PubMed PMID: 10021489). Reducing the diameter of the liposome used in the Postma et al. publication creates the problem of too low encapsulation of the chelating agent and loss of too much chelating agent in manufacturing the liposomes. Thus none of the prior publications by themselves or taken together describes how to create a liposome encapsulated chelating agent formulation that would be suitable to treat patients with iron overload. The invention described here overcomes the limitations of the prior publications.

In U.S. Pat. No. 4,397,867 (Treatment of arthritic complaints), David R. Blake, the inventor, discloses using a liposome to deliver the chelating agent to reduce joint inflammation but provides no instructions on how to prepare small diameter liposomes with a high concentration of deferoxamine. Indeed in the description, the non-encapsulated deferoxamine had to be removed from the liposome by a tedious centrifugation and re-suspension procedure that was repeated five times. The method described herein avoids the loss of the deferoxamine by encapsulation more so an excessive amount of chelating agent is not lost in the extensive separation process required to prepare the encapsulated chelating agent.

In US Patent Application Pub. No. 2005/0175684 A1, a targeted iron chelating agent delivery system that comprises an iron chelating agent, a targeting agent and a lipid carrier, e.g., a liposome. In a similar vein U.S. Pat. No. 8,029,795 B2 describes a targeted iron chelating agent delivery system that comprises an iron chelating agent, a targeting agent and a lipid carrier, e.g., a liposome. However, the methods proposed to prepare the liposome encapsulated iron chelating agent do not describe a high efficiency encapsulation procedure or a high chelating agent to lipid ratio and require the deferoxamine to be present when the liposomes are initially prepared. The methods described in the present invention load the chelating agent into the liposome after the liposome is formed, provide a high chelating agent to lipid ratio and a highly efficient loading process so that the expensive chelating agents such as deferoxamine or deferasirox are not wasted during the encapsulation process.

Certain hydrophilic or amphiphilic compounds can be loaded into preformed liposomes using transmembrane pH- or ion-gradients (Zucker et al., 2009). This technique is called active or remote loading. Compounds amenable to active loading generally have a molecular weight under 500, are water-soluble, are able to change from an uncharged form, which can diffuse across the liposomal membrane, to a charged form that is not capable thereof (Zucker et al., 2009). Typically, the functional compound is loaded by adding it to a suspension of liposomes prepared to have a lower outside/higher inside pH- or ion-gradient. Using active loading, a high functional-compound-to-lipid mass ratio and a high loading efficiency (up to 100%) can be achieved. Examples are active loading of anticancer drugs doxorubicin, daunorubicin, and vincristine (Cullis et al., 1997, and references therein).

To date, a pharmaceutical formulation of chelating agents has not been developed utilizing active loading of the aqueous core of a liposome with a high molecular weight chelating agent such as deferoxamine (MW 561) or a sparingly soluble chelating agent (solubility less than 2 mg/mL) such as deferasirox (MW 373). Thus, in an exemplary embodiment, the presenting invention provides a pharmaceutical formulation of deferoxamine that is stably entrapped within a preformed liposome that contains an ammonium salt and requires an enhancing reagent such as ethanol be present in order for the remote loading to occur. The invention also provides a pharmaceutical formulation for the encapsulation of the sparingly water-soluble iron chelating agent, i.e., deferasirox into the interior aqueous medium of a preformed liposome from a precipitated formed from adding the deferasirox DMSO solution to the preformed liposome containing a divalent acetate salt. When the deferasirox DMSO solution is added to the liposome, the deferasirox precipitates and the chelating agent is transferred from the precipitate into the liposome and retained as a divalent salt. To date no one has reported on the encapsulation of deferasirox into a liposome. The encapsulation of two or more chelating agents in the same liposome to serve as a universal treatment for patients that have been exposed to metals or radionuclides such as uranium and plutonium is not currently described in the literature. The new formulations represent a significant advance in controlling the efficiency of loading and concentration of chelating agents such as deferoxamine and deferasirox in unilamellar liposomes with diameters less than 300 nanometers. The invention provides formulations with a high chelating agent to lipid ratio. This makes the liposomal chelating agents suitable for administration as a parenteral metal chelation therapy in mammals.

### SUMMARY OF THE INVENTION

In various embodiments, the invention provides a metal removal treatment system comprising a mixture of a metal chelating agent on the inside of the lipid vesicle as a salt of a multivalent ion. Furthermore, in various embodiments, the concentration of the metal chelating agent inside the lipid vesicle is greater than about 200 mM and the diameter of the lipid vesicle is equal to or less than about 300 nm. The term “lipid vesicle,” as used herein, includes a carrier comprising lipid molecules, e.g., a liposome. The metal chelating agent and the liposome encapsulated metal chelating agent of the metal removal treatment system of the present invention can be combined in various ways. For example, the chelating agent outside of the liposome can be at a low percentage, e.g., less than or equal to about 30% of the total chelating agent concentration in the system so that the system mainly removes metal from inside of cells of the RES. In another embodiment, two liposome preparations with different metal chelating agent salt combinations inside the liposome can be mixed together so that the most attractive features of both chelating agents are exploited to remove metal from a patient. In a third embodiment, one or more chelating agents can be used to remote load a second/third chelating agent; this provides another approach to obtaining the best characteristics of multiple chelating agents and enables the removal of more than one metal with one formulation.

Exemplary advantages of the metal chelating agent delivery system of the present application include: (1) An exceptionally high concentration of the chelating agent inside of the liposome so that the total dose of lipid administered to patients is much lower than previously described for prior liposome metal chelating agent preparations. (2) high efficiency encapsulation of the chelating agent so the process is cost effective; (3) delivery of the metal chelating agent to the liver and spleen without significant loss of the chelating agent via renal clearance. This increases the efficiency of metal removal hence (4) the formulations of the invention reduce the amount of chelating agent needed and (5) they provide a prolonged duration of treatment, thus, reducing the frequency of dosing required to obtain a therapeutic effect. A sixth advantage is that counter ions such as zinc, magnesium or calcium can be included in the liposome to remedy the well known tendency of metal chelating agents to remove such endogenous metals from the body.

Exemplary soluble metal chelating agents of use in the formulations and methods of the present invention include, for example: ethylenediamine tetracetic acid (EDTA) also known as ethylenediamine tetraacetic acid (calcium disodium versante), diethylenetriaminepentaacetic acid (DTPA), deferoxamine, deferiprone, pyridoxal isonicotinoyl hydrazone, rhodotorulic acid, picolinic acid, nicotinic acid, neoaspergillic acid, methionine, lactic acid, N,N-ethylene bis[N-phosphonomethyl]glycine, tetraethylenepentaamine heptaacetic acid (TPHA), tri(2-aminoethyl)aminehexaacetic acid (TAAHA), triethylenetetraaminehexaacetic acid (TTHA), oxybis(ethylenenitrilo)tetraacetic acid (BAETA), trans-1,2-cyclohexaneediaminetetraacetic acid, salicyclic acid, tartaric acid, 2,3-dihydroxybenzoic acid, penicillamine, etidronic acid (1-hydroxyethan-1,1-diyl)bis(phosphonic acid), dimercaptosuccinic acid, dimercapto-propane sulfonate, and dimercaprol, desferrithiocin (DFT), polycarboxylates, hydroxamates, catecholates, hydroxypyridonates, terathalamides and analogues or derivatives of each. Exemplary sparingly soluble chelating agents of use in the formulations and methods of the present invention include: deferasirox, HBED (N,N′-bis(2-hydroxbenzyl)ethylenediamine-N—N-diacetic acid) and HBPD (N,N′-bis(2-hydroxybenyzyl)propylene-1,3-diamine-N,N′-diacetic acid.

Exemplary lipid carriers of use in the methods and formulations of the present invention include, for example, liposomes, e.g., unilamellar and multilamellar liposomes, as well as, phospholipid and nonphospholipid liposomes.

In one embodiment, the concentration of the metal chelating agent within the metal removal treatment system is from about 200 mM up to about 1 M. In various embodiments, the diameter of the liposome is approximately equal to or greater than about 30 nanometers to about 300 nanometers. In an exemplary embodiment the fraction of the chelating agent within the lipid vesicle is equal to at least 40%, at least 50%, at least 70%, at least 85% and at least 98% of the total amount of chelating agent in the mixture used to prepare the formulation. In a preferred embodiment, the pharmaceutical formulation of the metal removal treatment system of the invention includes a lipid vesicle with a diameter of from about 50 nanometers to about 200 nanometers. In various embodiments, the pharmaceutical formulation of the invention has a chelating agent to lipid ratio of about 0.5 mole chelating agent/mole of lipid and up to about 95% of the chelating agent is contained in the aqueous space of the lipid vesicle.

The present invention is also drawn to methods for preparing the metal removal treatment system of the invention. An exemplary method includes one or more of the steps of combining a lipid carrier containing a high concentration of an ammonium or multivalent salt on the inside with the metal chelating agent on the outside and allowing the metal chelating agent to be accumulate on the inside of the liposome as a chelating agent-salt complex.

The invention also provides methods for treating metal-overload in a mammal in need of such treatment, comprising administering to the mammal a metal removal treatment system, e.g., an metal chelating agent encapsulated inside of a lipid vesicle, e.g., a liposome, are also provided. In a preferred embodiment, the metal chelating agent delivery system is administered so as to accumulate in the bone marrow, spleen and liver. Prior to administration, the metal chelating agent drug delivery system can be suspended or diluted in a pharmaceutically acceptable excipient or carrier, e.g., saline, dextrose or water.

Other embodiments, objects and advantages are set forth in the Detailed Description that follows.

### BRIEF DESCRIPTION OF THE DRAWINGS

FIG. 1 illustrates the pathways for iron recycling.

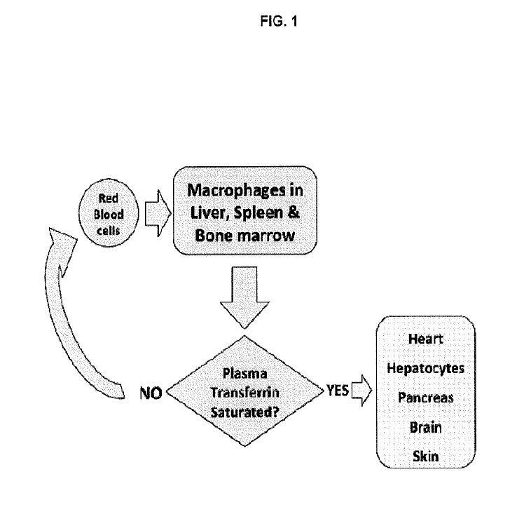

FIG. 2 illustrates representative iron chelating agents.

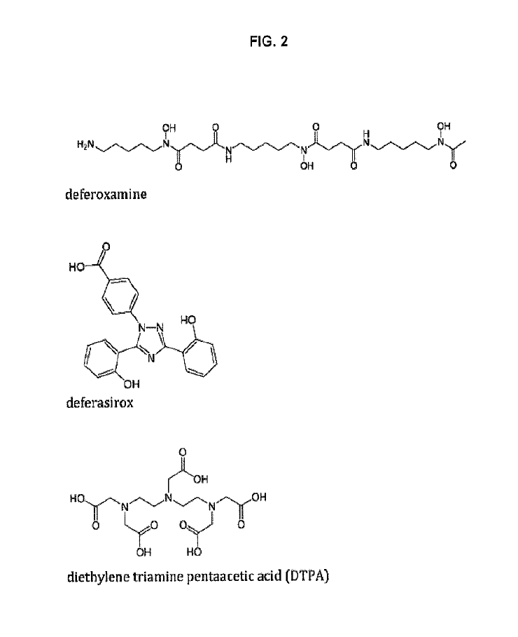

FIG. 3 illustrates the diameters and morphology of various types of liposomes.

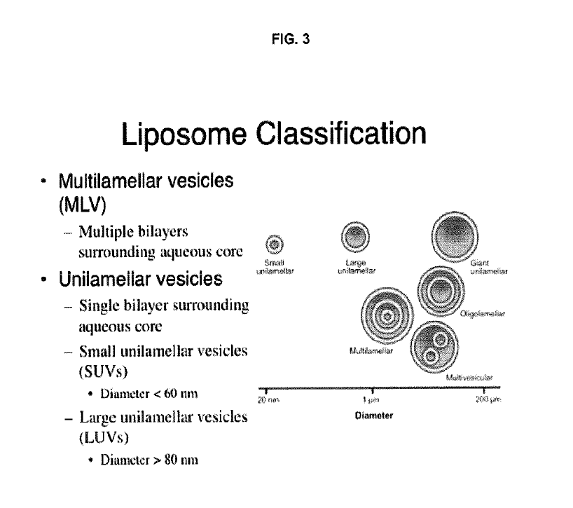

FIG. 4 illustrates a standard curve of DFO measured in the presence of 5% Triton X-100 in 50 mM HCL and 2 mM FeCL3 at 468 nm.

FIG. 5 illustrates a standard curve of DFO measured by HPLC

FIG. 6 illustrates a standard curve for DOPC and cholesterol measured by HPLC using detection at 205 nm.

FIG. 7 illustrates DOPC/Chol (3/0.5 mol/mol) liposomes containing 250 mM ammonium sulfate which were incubated with DFO at 200 g/mol and varying amounts of 1-butanol was added. The solution pH was adjusted to 8 and the liposomes heated at 45° C. for 20 min. to initiate loading.

FIG. 8 illustrates POPC/Chol (3/0.5 mol/mol) liposomes containing 250 mM ammonium sulfate which were incubated with DFO at 200 g/mol and varying amounts of ethanol was added. The solution pH was adjusted to 8 and the liposomes heated at 45° C. for 20 min. to initiate loading.

FIG. 9 illustrates the D/L ratio and loading efficiency of DFO into DOPC/Chol (3/0.5 mol/mol) liposomes as a function of loading time.

FIG. 10 illustrates the loading efficiency of DFO into DOPC/Chol (3/0.5 mol/mol) liposomes as a function of temperature.

FIG. 11 illustrates the loading efficiency of DFO into POPC/Chol/DSPG (3/0.5/0.15 mol/mol/mol) liposomes as a function of loading temperature.

FIG. 12 illustrates the loading efficiency of DFO into POPC/Chol (3/0.5 mol/mol/mol) liposomes as a function of loading temperature.

FIG. 13 illustrates the D/L ratio and loading efficiency of DFO into DOPC/Chol (3/0.5 mol/mol) liposomes as a function of pH.

FIG. 14 illustrates the measured D/L ratio and resultant loading efficiency of DOPC/Chol (3/0.5 mol/mol) ammonium sulfate containing liposomes loaded with DFO.

FIG. 15 illustrates the measured D/L ratio and resultant loading efficiency of DOPC/Chol (3/0.5 mol/mol) ammonium sulfate (500 mM sulfate) containing liposomes loaded with DFO.

FIG. 16 illustrates the loading efficiency of DFO by remote loading into DOPC/Chol (3/0.5 mol/mol) liposomes containing increasing concentrations of internal sulfate.

FIG. 17 illustrates liposome formulations composed of DOPC/Chol (3/0.5 mol/mol) containing ammonium DTPA (0.5M acetate) which were incubated with DFO at a drug to lipid ratio (D/L) of 500 g drug/mol of phospholipid in the presence of varying amounts of ethanol using conditions described below. The liposomes were purified from unencapsulated chelating agent, chelating agent and lipid were measured and the resultant chelating agent to lipid ratio plotted against % ethanol (v/v).

FIG. 18 illustrates the loading efficiency of liposomes composed of DOPC/Chol (3/0.5 mol/mol) containing TEA Dextran sulfate (0.5M SO₄ equivalents) as a function of input drug and lipid ratio.

FIG. 19 illustrates DFO loading into liposome formulations composed of DOPC/Chol (3/0.5 mol/mol) containing ammonium either ammonium sulfate or a mixture of ammonium sulfate and zinc sulfate as a function of time.

FIG. 20 illustrates the effect of different alcohols on the remote loading of DFO.

FIG. 21 illustrates the effect of DFO concentration in the loading solution during remote loading.

FIG. 22 illustrates the temperature effect on DFO active loading into liposomes.

FIG. 23 illustrates the temperature effect on DFO and DOX active loading into liposomes.

FIG. 24 illustrates the standard curve for deferasirox measured by HPLC using detection at 254 nm.

FIG. 25 illustrates liposome formulations composed of HSPC/Chol or POPC/Chol containing either sodium sulfate or ammonium sulfate which were incubated with deferasirox at a drug to lipid ratio of 100 g drug/mol of phospholipid using conditions described below. The liposomes were purified from unencapsulated drug and the efficiency of deferasirox encapsulation within the liposomes is shown, expressed as % of added drug.

FIG. 26 illustrates liposome formulations composed of POPC/Chol containing either 120 mM calcium acetate or 250 mM calcium acetate which were incubated with deferasirox at a drug to lipid ratio of 100 and 200 g drug/mol of phospholipid using conditions described below. The liposomes were purified from unencapsulated drug and the efficiency of deferasirox encapsulation within the liposomes is shown, expressed as encapsulated drug (g drug/mol phospholipid).

FIG. 27 illustrates liposome formulations composed of POPC/Chol containing either 120 mM calcium acetate, 120 mM zinc acetate, or 250 mM magnesium acetate which were incubated with deferasirox at a chelating agent to lipid ratio of 100, 150 and 200 g drug/mol of phospholipid using conditions described below. The liposomes were purified from unencapsulated chelating agent and the efficiency of deferasirox encapsulation within the liposomes is shown, expressed as encapsulated chelating agent (g chelating agent/mol phospholipid).

FIG. 28 illustrates PK of DFO administered to mice when formulated as LDFO-1, LDFO-2, LDFO-4 or as the unencapsulated DFO, Desferal (n=3). The t-test P values comparing LDFO-1 to LDFO-2 and LDFO-4 to LDFO-1 or -2 are shown for the 24 h time point. DFO was undetectable when dosed from Desferal at 24 h.

FIG. 29 illustrates the percent of the injected dose (% ID) remaining in blood of Liposomal DFO (LDFO) and LDFO that was also loaded with four different divalent cations after the DFO was loaded in the liposomes: barium, copper, magnesium or zinc.

FIG. 30 illustrates Fe-59 urine cumulative excretion after IV dosing with the listed treatment groups (each data point is urine pooled from 7 animals).

FIG. 31 illustrates a comparison of iron removal form the liver of mice that were overloaded with iron dextran. Groups from left to right are: Vehicle, non-encapuslated DFO, LDFO encapsulated using ammonium sulfate, LDFO encapsulated with the ammonium ascorbate phosphate and non-treated non-overloaded healthy mice. Results are presented as the mean and standard deviation of 4 mice per group.

### DESCRIPTION OF THE PREFERRED EMBODIMENTS

### Introduction

In utilizing liposomes for delivery of functional compounds, it is generally desirable to load the liposomes to high concentration, resulting in a high agent-to-lipid mass ratio, since this reduces the amount of liposomes to be administered per treatment to attain the required therapeutic effect of the agent, all the more since several lipids used in liposomes have a dose-limiting toxicity by themselves. The loading efficiency is also of importance for cost considerations, since poor loading results in an increase loss of agent during the manufacture of the liposome encapsulated chelating agent.

The present invention provides liposomes encapsulating metal chelating agents, methods of making such liposomes and pharmaceutical formulations containing such liposomes of the invention.

In various embodiments the present invention provides metal decorporation systems in which the final chelating agent-to-lipid ratio for high molecular weight chelating agents such as deferoxamine, that do not readily cross the liposome membrane, are greatly increased over those in the art. In various embodiments, the ratio is optimized by adding a membrane modifier, e.g., ethanol, under specified conditions which enables for the first time, the remote loading of exemplary metal chelating agents, e.g., deferoxamine into the liposome. In an exemplary embodiment, the decorporation system is appropriate for administration to a mammalian subject to remove excess metal ion in the subject.

The present invention also provides methods for increasing the final agent-to-lipid ratio for chelating agents that are sparingly soluble in water. For example, the chelating agent:lipid ratio of chelating agents such as deferasirox can be increased by adding the deferasirox solubilized in a polar aprotic solvent such as acetone, acetonitrile, N,N′-dimethylformamide, dimethylacetamide, dioxane, dimethylsulfoxide (DMSO), ethylacetate, hexamethylphosphorotriamide, glyme (dimethylethoxyethane), N-methyl-2-pyrrolidone, sulfolane, or tetrahydrofuran to the liposome in an aqueous milieu containing a high concentration of a divalent salt, e.g., a cation acetate solution. A deferasirox precipitate is formed but then the deferasirox is transferred into the liposome and the precipitate disappears.

In an exemplary embodiment, the invention provides a pharmaceutical formulation comprising a liposome having a bilayer of lipids encapsulating an aqueous compartment. Encapsulated within the aqueous compartment is the metal chelating agent and a salt of a remote loading agent. In various embodiments, about 30%, about 40%, about 50%, about 70%, about 90% or about 98% of the sparingly water-soluble agent originally external to the liposome is encapsulated within the aqueous compartment of the liposome.

In an exemplary embodiment, the agent is deferoxamine and at least about 30% of the deferoxamine originally external to the liposome is taken up by the liposome.

### Liposomes

The term liposome is used herein in accordance with its usual meaning, referring to microscopic lipid vesicles composed of a bilayer of phospholipids or any similar amphipathic lipids encapsulating an internal aqueous medium. The liposomes of the present invention can be unilamellar vesicles such as small unilamellar vesicles (SUVs) and large unilamellar vesicles (LUVs), and smaller multilamellar vesicles (MLV), typically varying in size from 50 nm to 300 nm. No particular limitation is imposed on the liposomal membrane structure in the present invention. The term liposomal membrane refers to the bilayer of phospholipids separating the internal aqueous medium from the external aqueous medium.

Exemplary liposomal membranes useful in the current invention may be formed from a variety of vesicle-forming lipids, typically including dialiphatic chain lipids, such as phospholipids, diglycerides, dialiphatic glycolipids, egg sphingomyelin and glycosphingolipid, cholesterol and derivatives thereof, and combinations thereof. As defined herein, phospholipids are amphiphilic agents having hydrophobic groups formed of long-chain alkyl chains, and a hydrophilic group containing a phosphate moiety. The group of phospholipids includes phosphatidic acid, phosphatidyl glycerols, phosphatidylcholines, phosphatidylethanolamines, phosphatidylinositols, phosphatidylserines, and mixtures thereof. Preferably, the phospholipids are chosen from egg yolk phosphatidylcholine (EYPC), soy phosphatidylcholine (SPC), palmitoyl-oleoyl phosphatidylcholine, dioleyl phosphatidylcholine, 1,2-dipalmitoyl-sn-glycero-3-phosphocholine (DPPC), 1,2-dimyristoyl-sn-phosphatidylcholine (DMPC), hydrogenated soy phosphatidylcholine (HSPC), distearoyl phosphatidylcholine (DSPC), or hydrogenated egg yolk phosphatidylcholine (HEPC), egg phosphatidylglycerol, distearoylphosphatidylglycerol (DSPG), sterol modified lipids, cationic lipids and zwitterlipids

In the method according to the present invention, an exemplary liposomal phase transition temperature is between about −20° C. and about 100° C., e.g., between about −20° C. and about 65° C. The phase transition temperature is the temperature required to induce a change in the physical state of the lipids constituting the liposome, from the ordered gel phase, where the hydrocarbon chains are fully extended and closely packed, to the disordered liquid crystalline phase, where the hydrocarbon chains are randomly oriented and fluid. Above the phase transition temperature of the liposome, the permeability of the liposomal membrane increases. Choosing a high transition temperature, where the liposome would always be in the gel state, could provide a non-leaking liposomal composition, i.e. the concentration of the sparingly water-soluble agent in the internal aqueous medium is maintained during exposure to the environment. Alternatively, a liposome with a transition temperature between the starting and ending temperature of the environment it is exposed to provides a means to release the sparingly water-soluble agent when the liposome passes through its transition temperature. Thus, the process temperature for the active-loading technique typically is above the liposomal phase transition temperature to facilitate the active-loading process. As is generally known in the art, phase transition temperatures of liposomes can, among other parameters, be influenced by the choice of phospholipids and by the addition of steroids like cholesterol, lanosterol, cholestanol, stigmasterol, ergosterol, and the like. Hence, in an embodiment of the invention, a method according to any of the foregoing is provided in which the liposomes comprise one or more components selected from different phospholipids and cholesterol in several molar ratios in order to modify the transition, the required process temperature and the liposome stability in plasma. Less cholesterol in the mixture will result in less stable liposomes in plasma. An exemplary phospholipid composition of use in the invention comprises between about 10 and about 50 mol % of steroids, preferably cholesterol.

In accordance with the invention, liposomes can be prepared by any of the techniques now known or subsequently developed for preparing liposomes. For example, the liposomes can be formed by the conventional technique for preparing multilamellar lipid vesicles (MLVs), that is, by depositing one or more selected lipids on the inside walls of a suitable vessel by dissolving the lipids in chloroform and then evaporating the chloroform, and by then adding the aqueous solution which is to be encapsulated to the vessel, allowing the aqueous solution to hydrate the lipid, and swirling or vortexing the resulting lipid suspension. This process engenders a mixture including the desired liposomes. Alternatively, techniques used for producing large unilamellar lipid vesicles (LUVs), such as reverse-phase evaporation, infusion procedures, and detergent dilution, can be used to produce the liposomes. A review of these and other methods for producing lipid vesicles can be found in the book (Liposome Technology: Liposome preparation and related Techniques, 3^{rd} addition, 2006, G. Gregoriadis, ed.) which is incorporated herein by reference. For example, the lipid-containing particles can be in the form of steroidal lipid vesicles, stable plurilamellar lipid vesicles (SPLVs), monophasic vesicles (MPVs), or lipid matrix carriers (LMCs). In the case of MLVs, if desired, the liposomes can be subjected to multiple (five or more) freeze-thaw cycles to enhance their trapped volumes and trapping efficiencies and to provide a more uniform interlamellar distribution of solute.

Following liposome preparation, the liposomes are optionally sized to achieve a desired size range and relatively narrow distribution of liposome sizes. A size range of from about 30 to about 200 nanometers allows the liposome suspension to be sterilized by filtration through a conventional sterile filter, typically a 0.22 micron or 0.4 micron filter. The filter sterilization method can be carried out on a high throughput basis if the liposomes have been sized down to about 20-300 nanometers. Several techniques are available for sizing liposomes to a desired size. Sonicating a liposome suspension either by bath or probe sonication produces a progressive size reduction down to small unilamellar vesicles less than about 50 nanometer in size. Homogenization is another method which relies on shearing energy to fragment large liposomes into smaller ones. In a typical homogenization procedure, multilamellar vesicles are recirculated through a standard emulsion homogenizer until selected liposome sizes, typically between about 50 and 500 nanometers, are observed. In both methods, the particle size distribution can be monitored by conventional laser-beam particle size determination. Extrusion of liposome through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes, to achieve a gradual reduction in liposome size. Other useful sizing methods such as sonication, solvent vaporization or reverse phase evaporation are known to those of skill in the art.

Exemplary liposomes for use in various embodiments of the invention have a size of from about 30 nm to about 300 nm, e.g., from about 50 nm to about 250 nm.

The internal aqueous medium, as referred to herein, typically is the original medium in which the liposomes were prepared and which initially becomes encapsulated upon formation of the liposome. In accordance with the present invention, freshly prepared liposomes encapsulating the original aqueous medium can be used directly for active loading. Embodiments are also envisaged however wherein the liposomes, after preparation, are dehydrated, e.g. for storage. In such embodiments the present process may involve addition of the dehydrated liposomes directly to the external aqueous medium used to create the transmembrane gradients. However it is also possible to hydrate the liposomes in another external medium first, as will be understood by those skilled in the art. Liposomes are optionally dehydrated under reduced pressure using standard freeze-drying equipment or equivalent apparatus. In various embodiments, the liposomes and their surrounding medium are frozen in liquid nitrogen before being dehydrated and placed under reduced pressure. To ensure that the liposomes will survive the dehydration process without losing a substantial portion of their internal contents, one or more protective sugars are typically employed to interact with the lipid vesicle membranes and keep them intact as the water in the system is removed. A variety of sugars can be used, including such sugars as trehalose, maltose, sucrose, glucose, lactose, and dextran. In general, disaccharide sugars have been found to work better than monosaccharide sugars, with the disaccharide sugars trehalose and sucrose being most effective. Typically, one or more sugars are included as part of either the internal or external media of the lipid vesicles. Most preferably, the sugars are included in both the internal and external media so that they can interact with both the inside and outside surfaces of the liposomes' membranes. Inclusion in the internal medium is accomplished by adding the sugar or sugars to the buffer which becomes encapsulated in the lipid vesicles during the liposome formation process. In addition to the sugars, a co-lyophilization agent such as glycine, betaine or carnitine, can be included to further increase the stability of the lyophilized liposome chelators. In these embodiments the external medium used during the active loading process should also preferably include one or more of the protective sugars

As is generally known to those skilled in the art, polyethylene glycol (PEG)-lipid conjugates have been used extensively to improve circulation times for liposome-encapsulated functional compounds, to avoid or reduce premature leakage of the functional compound from the liposomal composition and to avoid detection of liposomes by the body's immune system. Attachment of PEG-derived lipids onto liposomes is called PEGylation. Hence, in one embodiment of the invention, the liposomes are PEGylated liposomes. Suitable PEG-derived lipids according to the invention, include conjugates of DSPE-PEG, functionalized with one of carboxylic acids, glutathione (GSH), maleimides (MAL), 3-(2-pyridyldithio) propionic acid (PDP), cyanur, azides, amines, biotin or folate, in which the molecular weight of PEG is between 2000 and 5000 g/mol. Other suitable PEG-derived lipids are mPEGs conjugated with ceramide, having either C₈- or C₁₆-tails, in which the molecular weight of mPEG is between 750 and 5000 daltons. Still other appropriate ligands are mPEGs or functionalized PEGs conjugated with glycerophospholipds like 1,2-dimyristoyl-sn-glycero-3-phosphoethanolamine (DMPE), 1,2-dipalmitoyl-sn-glycero-3-phosphoethanolamine (DPPE), 1,2-dioleoyl-sn-glycero-3-phosphoethanolamine (DOPE) and 1,2-distearoyl-sn-glycero-3-phosphoethanolamine (DSPE), and the like. PEGylation of liposomes is a technique generally known by those skilled in the art.

In various embodiments, the liposomes are PEGylated with DSPE-mPEG conjugates (wherein the molecular weight of PEG is typically within the range of 750-5000 daltons, e.g. 2000 daltons). The phospholipid composition of an exemplary PEGylated lipsome of the invention may comprise up to 0.8-20 mol % of PEG-lipid conjugates.

Targeting mechanisms generally require that the targeting agents be positioned on the surface of the liposome in such a manner that the target moieties are available for interaction with the target, for example, a cell surface receptor. In an exemplary embodiment, the liposome is manufactured to include a connector portion incorporated into the membrane at the time of forming the membrane. An exemplary connector portion has a lipophilic portion which is firmly embedded and anchored in the membrane. An exemplary connector portion also includes a hydrophilic portion which is chemically available on the aqueous surface of the liposome. The hydrophilic portion is selected so that it will be chemically suitable to form a stable chemical bond with the targeting agent, which is added later. Techniques for incorporating a targeting moiety in the liposomal membrane are generally known in the art.

### Water-Soluble Chelating Agents

Exemplary water-soluble chelating agents of use in the methods and formulations of the invention include chelators with a solubility in water of at least about 1.9 mg/mL (e.g., at ambient temperature, which is typically about 20° C., and pH=7). These chelating agents include ethylenediamine tetraacetic acid (EDTA) also known as ethylenediamine tetraacetic acid (calcium disodium versante), diethylenetriaminepentaacetic acid (DTPA), deferoxamine, pyridoxal isonicotinoyl hydrazone, rhodotorulic acid, penicillamine etidronic acid (1-hydroxyethan-1,1-diyl)bis(phosphonic acid), dimercaptosuccinic acid, dimercapto-propane sulfonate, and dimercaprol. This list of agents, however, is not intended to limit the scope of the invention. In fact, the functional chelating agent can be any sparingly water-soluble amphipathic weak base chelating agent or amphipathic weak acid chelating agent or a water-soluble chelating agent. Embodiments wherein the water-soluble chelating agent is not a pharmaceutical or medicinal agent are also encompassed by the present invention. As indicated above, the present invention provides liposomes encapsulating a complex between a water-soluble chelating agent and a multivalent salt. In an exemplary embodiment, the chelating agent is loaded into the liposome in an uncomplexed salt form, as a metal ion complex or as a combination of a salt metal ion complex.

### Sparingly Water-Soluble Chelating Agents

In various embodiments, the present invention also provides liposomes encapsulating a complex between a sparingly water-soluble agent and a multivalent salt. In the context of the present invention the term sparingly water-soluble means being insoluble or having a very limited solubility in water, more in particular having an aqueous solubility of less than 1.9 mg/mL at ambient temperature, which is typically about 20° C., and pH=7, e.g., having an aqueous solubility of less than about 1.5 mg/mL, less than about 1.2 mg/mL, less than about 1 mg/mL, less than about 0.8 mg/ml, less than about 0.5 mg/mL or less than about 0.3 mg/mL. The functional chelating agent can be any sparingly water-soluble amphipathic weak base chelator or amphipathic weak acid chelating agent or a water-soluble chelating agent. Embodiments wherein the water-soluble chelating agent is not a pharmaceutical or medicinal agent are also encompassed by the present invention. One such sparingly soluble chelating agent is deferasirox others include HBED (N,N′-bis(2-hydroxbenzyl)ethylenediamine-N—N-diacetic acid) and HBPD (N,N′-bis(2-hydroxybenyzyl)propylene-1,3-diamine-N,N′-diacetic acid.

Exemplary sparingly water-soluble amphipathic weak bases (chelating agents) of use in the invention have an octanol-water distribution coefficient (log D) at pH 7 between about −2.5 and about 7 and pKa<11, while sparingly water-soluble amphipathic weak acids have a log D at pH 7 between about −2.5 and about 7 and pKa>3. Preferably, the sparingly water-soluble agents to be actively loaded have good thermal stability (to about 70° C. for 4 hours) and good chemical stability at higher (7-11) or lower (4-7) pH.

Typically, the terms weak base and weak acid (chelating agents), as used in the foregoing, respectively refer to compounds that are only partially protonated or deprotonated in water. Examples of protonable agents include compounds having an amino group, which can be protonated in acidic media, and compounds which are zwitterionic in neutral media and which can also be protonated in acidic environments. Examples of deprotonable agents include compounds having a carboxy group, which can be deprotonated in alkaline media, and compounds which are zwitterionic in neutral media and which can also be deprotonated in alkaline environments.

The term zwitterionic refers to compounds that can simultaneously carry a positive and a negative electrical charge on different atoms. The term amphipathic, as used in the foregoing is typically employed to refer to compounds having both lipophilic and hydrophilic moieties. The foregoing implies that aqueous solutions of compounds being weak amphipathic acids or bases simultaneously comprise charged and uncharged forms of said compounds. Only the uncharged forms may be able to cross the liposomal membrane.

When agents of use in the present invention contain relatively basic or acidic functionalities, salts of such compounds are included in the scope of the invention. Salts can be obtained by contacting the neutral form of such compounds with a sufficient amount of the desired acid or base, either neat or in a suitable inert solvent. Examples of salts for relative acidic compounds of the invention include ammonium, sodium, potassium, calcium, copper, cobalt, magnesium, manganese, nickel, zinc, ammonium, or organic amino salts, or a similar salt. When compounds of the present invention contain relatively basic functionalities, acid addition salts can be obtained by contacting the neutral form of such compounds with a sufficient amount of the desired acid, either neat or in a suitable inert solvent. Examples of acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, or polyglycerol sulfate, polyglycerol phosphate and the like. Also included are salts of amino acids such as arginate and the like, and salts of organic acids like glucuronic or galactunoric acids and the like. Also included are: polymers such as: dextrin sulfate, dextran sulfate, heparin, maltodextrin sulfate, sulfobutylether cyclodextrin, polyethyleneimine, polyamidoamine dendrimers, the carboxylate version of polyamidoamine dendrimers, hyaluronic acid, polyphosphoric acid. Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

### Active Loading

As indicated above, in an exemplary embodiment, the pre-formed liposomes are loaded with the sparingly water-soluble chelating agent that is precipitated from an aprotic solution and combined with the liposome that is used in an active or remote loading technique. The process of active loading, involves the use of transmembrane potentials. The principle of active loading, in general, has been described extensively in the art. The terms active-loading and remote-loading are synonymous and will be used interchangeably.

During active loading, the precipitated sparingly water-soluble agent is transferred from the external aqueous medium across the liposomal membrane to the internal aqueous medium by a transmembrane proton- or ion-gradient. Alternative the water-soluble chelating agent in the presence of a membrane modifier, such as ethanol, can be loaded into the interior of the liposome. The term gradient of a particular compound as used herein refers to a discontinuous increase of the concentration of said compound across the liposomal membrane from outside (external aqueous medium) to inside the liposome (internal aqueous medium).

To create the concentration gradient, the liposomes are typically formed in a first liquid phase containing the multivalent salt, typically aqueous, followed by replacing or diluting the first liquid phase with a second liquid phase such as 0.3 M sucrose, so that the concentration of multivalent salt is reduced and a salt concentration gradient (inside salt concentration is high salt/outside salt concentration is low). The diluted or new external medium has a different concentration of the charged species or a totally different charged species, thereby establishing the ion- or proton-gradient.

In an exemplary embodiment, the liposomes initially contain an active loading agent with a concentration of ammonium sulfate from about 250 mM to about 750 mM.

The replacement of the external medium can be accomplished by various techniques, such as, by passing the lipid vesicle preparation through a gel filtration column, e.g., a Sephadex or Sepharose column, which has been equilibrated with the new medium, or by centrifugation, dialysis, diafiltration or related techniques.

In an exemplary embodiment, the external buffer of the active loading system and chelating agent remaining external to the liposome after loading of the liposome are replaced with a physiologically compatible aqueous buffer. In various embodiments, the pH of the external buffer is from about 3.5 to about 8.0.

The efficiency of active-loading into liposomes depends, among other factors, on the chemical properties of the chelating agent to be loaded and the type and magnitude of the gradient applied across the liposome membrane. In an exemplary embodiment of the invention, a gradient is established across the liposomal membrane. The gradient is chosen from a pH-gradient, a sulfate-, phosphate-, citrate-, or acetate-salt gradient, an EDTA-ion gradient, an ammonium-salt gradient, an alkylated, e.g., methyl-, ethyl-, propyl- and amyl-, ammonium-salt gradient, a triethylammonium salt gradient, a Mn²⁺—, Co²⁺, Cu²⁺—, Na⁺—, K⁺, Ni²⁺, Zn²⁺, Ca²⁺, Mg²⁺ gradient, with or without using ionophores, or a combination thereof. These loading techniques have been extensively described in the art.

In an exemplary embodiment, the internal aqueous medium of pre-formed, i.e. unloaded, liposomes comprises a so-called active-loading buffer which contains water and, dependent on the type of gradient employed during active loading, may further comprise a sulfate-, phosphate-, citrate-, or acetate-salt, an ammonium-salt, an alkylated, e.g methyl-, ethyl-, propyl- and amyl, ammonium-salt, a Mn²⁺—, Co²⁺, Cu²⁺, Zn²⁺, Ca²⁺, Mg²⁺, Ni²⁺, or Na⁺/K⁺-salt, an EDTA-ion salt, and optionally a pH-buffer to maintain a pH-gradient. In an exemplary embodiment, the concentration of salts in the internal aqueous medium of the unloaded liposomes is between 50 and 1000 mM.

Exemplary amines of use in the present invention include, without limitation, trimethylammonium, triethylammonium, tributyl ammonium, diethylmethylammonium, diisopropylethyl ammonium, triisopropylammonium, N-methylmorpholinium, N-ethylmorpholinium, N-hydroxyethylpiperidinium, N-methylpyrrolidinium, N,N-dimethylpiperazinium, isopropylethylammonium, isopropylmethylammonium, diisopropylammonium, tert-butylethylammonium, dicychohexylammonium, protonized forms of morpholine, pyridine, piperidine, pyrrolidine, piperazine, imidazole, tert-bulylamine, 2-amino-2-methylpropanol-I,2-amino-2-methyl-propandiol-I,3, and tris-(hydroxyethyl)-aminomethane, diethyl-(2-hydroxyethyl)amine, tris-(hydroxymethyl)-aminomethane tetramethylammonium, tetraethylammonium, N-methylglucamine and tetrabutylammonium, polyethyleneimine, and polyamidoamine dendrimers.

Exemplary carboxylates of use in the invention include, without limitation, acetate, fumarte, pyruvate, lactate, citrate, diethylenetriaminepentaaceetate, melletic acetate, 1,2,3,4-butanetetracarboxylate, benzoate, isophalate, phthalate, 3,4-bis(carboxymethyl)cyclopentanecarboxylate, benzenetricarboxylates, benzenetetracarboxylates, ascorbate, glucuronate, and ulosonate.

Exemplary sulfates include, without limitation, sulfate, 1,5-naphthalenedisulfonate, dextran sulfate, sucrose octasulfate benzene sulfonate, poly(4-styrenesulfonate) trans resveratrol-trisulfate, sulfobutyletherbetacyclodextrn, polyglycerol sulfate, dextrin sulfate, maltodextrin sulfate and the like.

Exemplary phosphates and phosphonates include, but are not limited to: phosphate, ascorbyl phosphate, hexametaphosphate, phosphate glasses, polyphosphates, triphosphate, trimetaphosphate, bisphosphonates, ethanehydroxy bisphosphonate, octaphosphate tripentaerythritol, hexaphosphate dipentaerythritol, tetraphosphate pentaerythritol, pentaphosphate triglycerol polyglycerol phosphate and inositol hexaphosphate, ethanehydroxybisphosphonate and the like.

Exemplary salts may include one or more of a carboxylate, sulfate or phosphate including, but not limited to: 2-carboxybenensulfonate, creatine phosphate, phosphocholine, carnitine phosphate, and the carboxyl generation of polyamidoamines.

An exemplary external aqueous medium, used to establish the transmembrane gradient for active loading, comprises water, solubility enhancer, the sparingly water-soluble agent(s) to be loaded, and optionally sucrose to adjust the osmolarity and/or a chelating agent like EDTA to aid ionophore activity, more preferably sucrose and/or EDTA. Solutions of salts, e.g., saline may also be used to adjust osmolarity. Sucrose and other saccharides can also be used to adjust osmolarity. In an exemplary embodiment of the invention, a method for actively loading liposomes is provided wherein concentrations of the gradient-forming compound in the internal aqueous medium, and concentrations of the sparingly water-soluble agent(s) and solubility enhancer in the external medium are established of such magnitude that net transport of the sparingly water-soluble agent(s) across the liposomal membrane occurs during active loading.

In an exemplary embodiment, the transmembrane gradient is chosen from a pH-, ammonium sulfate- and magnesium acetate-gradient. As is generally known by those skilled in the art, transmembrane pH- (lower inside, higher outside pH) or calcium acetate-gradients can be used to actively load amphiphilic weak acids. Amphipathic weak bases can also be actively loaded into liposomes using an ammonium sulfate- or ammonium chloride-gradient.

Depending upon the permeability of the lipid vesicle membranes, the full transmembrane potential corresponding to the concentration gradient will either form spontaneously or a membrane transfer enhancing agent, e.g., an alcohol such as methanol, ethanol, propanol, tetiary butanol, or 2-(2-ethoxyethoxy)ethanol, a proton ionophore can be added to the medium. If desired, the membrane enhancing agent can be removed from the liposome preparation after loading of the chelating agent with the salt complex is complete, using chromatography, dialysis, diafiltration, evaporation or other separation techniques.

In an exemplary embodiment, the liposomes are exposed to a membrane transfer agent that is an alcohol, as set forth immediately above, at a concentration from about 0% v/v alcohol/aqueous buffer to about 20% v/v alcohol/aqueous buffer. A presently preferred alcohol is ethanol.

Typically the temperature of the medium during active loading is between about 0° C. and about 100° C., e.g., between about 0° C. and about 70° C., e.g., between about 4° C. and 65° C. In an exemplary embodiment, the loading temperature is from about 20° C. to about 120° C. In various embodiments, in which the chelating agent is deferoxamine, the loading temperature is from about 70° C. to about 120° C. In a further exemplary embodiment utilizing deferoxamine, the loading temperature is from about 80° C. to about 110° C.

The loading mixture is incubated for an appropriate period of time at a selected temperature. In various embodiments, the mixture is incubated from about 5 minutes to about 1 hour.

The encapsulation or loading efficiency, defined as encapsulated amount (e.g., as measured in moles) of the complex between the solubility enhancer and the sparingly water-soluble agent in the internal aqueous phase divided by the initial amount of moles of complex in the external aqueous phase multiplied by about 100%, is at least about 30%, preferably at least about 50%, at least about 60%, at least about 70% at least about 90% or at least about 98%. In an exemplary embodiment, the encapsulation efficiency is from about 50% to about 95% of the chelating agent used to prepare the liposome of the invention.

### Precipitate Enabling Solvents

As noted herein, in an exemplary embodiment of the invention a solution of the sparingly soluble chelating agent in a precipitate enabling solvent is added to the external aqueous medium of a liposome preparation and the precipitated chelating agent transfers from the external medium into the aqueous compartment of the liposome. Precipitate enabling solvents include, without limitation: polar aprotic solvents such as acetone, acetonitrile, N,N′ dimethylformamide, dimethylacetamide, dioxane, dimethylsulfoxide (DMSO), ethylacetate, hexamethylphosphorotriamide, glyme (dimethylethoxyethane), N-methyl-2-pyrrolidone, sulfolane, tetrahydrofuran and the like. The invention provides liposomes having sparingly water-soluble chelating agents encapsulated as a salt with the appropriate counterion within the aqueous compartment of a liposome.

According to an embodiment of the present invention, a method as defined in the foregoing is provided using a, co-solvent for the chelating agent. The co-solvent chelating agent solution typically forms a precipitate of the chelating agent when the sparingly water-soluble chelating agent is added to the external aqueous medium containing the liposome.

As will be apparent from the foregoing, the rate and efficiency of active-loading a given chelating agent into the liposome is controlled by varying one or more factors, including the transmembrane gradient, the choice of precipitation solvent, the choice of the membrane transfer enhancer the composition of the liposome membrane, the process temperature, etc. It is within the capabilities and the normal routine of those skilled in the art to adapt and optimize these parameters in conjunction to arrive at the most efficient process for a given sparingly water-soluble agent.

In various embodiments, the method of the invention makes use of a precipitation promoting solvent as described in the foregoing in the active-loading of liposomes to enhance the loading efficiency and/or rate of sparingly water-soluble agents. In various embodiments the loading enhancer is an aprotic solvent or an alcohol. As will be understood, exemplary embodiments involve combining the pre-formed liposomes, chelating agents, internal aqueous medium, external aqueous medium, gradients, etc. as defined in any of the foregoing. In an exemplary embodiment of the invention, the method includes combining the enhancing agent with the chelating agent in a first aqueous medium (i.e., the external medium defined hereinbefore) and contacting the resulting complex with liposomes encapsulating a second aqueous medium (i.e., the internal medium) under conditions appropriate for the complex to be transferred across the membrane and encapsulated essentially intact in the aqueous compartment.

In a preferred embodiment of the invention, the composition of the encapsulated chelating agent in the liposome has a chelating agent to-lipid mass ratio of at least about 1:15, e.g., at least about 1:10, e.g., at least about 1:5, e.g., at least about 1:4, at least about 1:2 or at least about 1:1.

In an exemplary embodiment, the chelating agent to-lipid mass ratio/mole lipid ratio is at least about 200 grams chelating agent to about 1 mole lipid, e.g., at least about 250 grams, at least about 400 grams, e.g., at least about 500 grams to about one mole of lipid. In an exemplary embodiment, the chelating agent is deferoxamine or deferasirox.

Typically, the liposomal pharmaceutical formulation comprises the chelating agent mainly in the form of a liposome encapsulated chelating agent and the chelating agent inside the liposome is with an appropriate salt. In an exemplary embodiment, the chelating agent on the outside constitutes less than 1/10 of chelating agent in the formulation. In an exemplary embodiment, about 98% or greater of the chelating agent is encapsulated in the aqueous compartment of the liposome and about 2% of the agent is located external to the liposome core.

Furthermore, in an exemplary embodiment, the amount of precipitation enabler in the internal aqueous medium of the agent loaded liposomes is significantly less than the ratio of chelating agent:precipitation enhancer in the solution to the liposome suspension prior to the loading of the sparingly water-soluble chelating agent into the liposome. In various embodiments, the stoichiometric ratio of enhancer:agent in the aqueous compartment of the final liposome-chealtor preparation is not more than about 5 mol %, e.g., not more than about 3 mol %, e.g., not more than about 1 mol %, e.g., not more than about 0.1 mol %, e.g., not more than about 0.01 mol % e.g., not more than about 0.001 mol % of the ratio in the complex prior to encapsulation of the sparingly water-soluble chelating agent or water-soluble chelating agent in the aqueous compartment of the liposome.

In one embodiment in which the liposome formulation is to be administered by intramuscular or subcutaneous injection, the liposomes are multivesicular (LMV) liposomes, e.g., about 300 nm in diameter. LMV are prepared by (a) hydrating a lipid film with an aqueous solution containing an amine salt of an anionic molecule, such as a solution of ammonium sulfate (e.g., about 250 mM), (b) homogenizing the resulting suspension to form a suspension of small unilamellar vesicles (SUV), and (c) freeze-thawing said suspension of SUV at about −20° C. repeating the freeze thaw cycle at least three times. The extraliposomal ammonium sulfate is then removed, e.g. by dialysis against about 0.15 M NaCl or about 300 mM sucrose. The LMV liposomes are then mixed with the enhancing agent and the chelating agent is added to the liposome. For the encapsulation of deferoxamine, preferably the internal salt complex contains a weakly basic moiety, and the suspension of LMV liposomes has a greater concentration of ammonium ions inside the liposomes than outside the liposomes. In an alternative implementation of this embodiment, the LMV encapsulate the sparingly soluble deferasirox as a divalent cation complex.

In another embodiment in which the liposome formulation is to be administered subcutaneously, intravenously or intra-arterially, unilamellar vesicles (UV) with a diameter between about 30 nm and about 200 nm are prepared by injection of a lipid solution in ethanol into an aqueous solution containing an amine salt of an anionic molecule, such as a solution of ammonium sulfate (e.g., about 250 mM) so that the concentration of ethanol is less than 30 v/v %. The resulting lipid dispersion is then extruded through polycarbonate membranes with a defined pore diameter of either, 50 nm, 100 nanometers (nm) or 200 nm. The ethanol and non-entrapped ammonium sulfate are removed from the UV suspension by dialysis in a dialysis cell against 300 mM sucrose 5 mM Tris buffer. The LUV which are extruded through 100 nm polycarbonate membranes have a diameter of approximately 100 nm are then mixed with a solution of the deferoxamine and the enhancing agents such as ethanol or at high temperature. The suspension of LUV has a greater concentration of ammonium ions inside the liposomes than outside the liposomes and the deferoxamine in the presence of the alcohol is able to concentrate inside the unilamellar vesicle to a higher concentration than it was on the outside of the UV.

In another embodiment the concentration of the LUV can encapsulate an acetate salt of sodium, zinc, copper, calcium or magnesium at about 300 mM. The acetate salt can be removed from outside of the LUV by dialysis against about 300 mM sucrose. A mixture of salts can be combined at a defined ratio such as 90:9:1/magnesium:zinc:copper to compensate for trace metals that may also be removed by the iron chelator (De Virgiliis et al., 1988).

The invention is further illustrated by reference to a specific embodiment in which deferoxamine is encapsulated in a liposome composed of phosphatidylcholine lipids and cholesterol and wherein the phosphatidylcholine:cholesterol mole ratio is between about 3 to about 2. The liposomes are from about 30 nm to about 300 nm in diameter. The deferoxamine is present in the liposome at a chelator (gram):lipid (mole) ratio of about 100 grams to about 1 mole. In an exemplary embodiment, these liposomes are suspended in an aqueous buffer having a pH of from about 5.5 to about 8.0.

The deferoxamine-loaded liposomes are, in an exemplary embodiment, prepared by a method comprising the following elements. The liposomes, which contain about 250 mM to about 750 mM ammonium sulfate as an active loading agent, are combined with the deferoxamine in an aqueous buffer. This mixture may then be combined with a membrane transfer agent, which is an alcohol, e.g., ethanol, at a concentration of from about 0% v/v alcohol/aqueous buffer to about 20% v/v alcohol/aqueous buffer. The mixture is incubated at from about 20° C. to about 120° C. for about 5 minutes to about 1 h. Exemplary liposomes prepared according to this method take up at least about 30% of the deferoxamine, which was originally external to the liposomes. The unencapsulated deferoxamine and loading buffer is replaced by a physiologically acceptable buffer.

In an exemplary embodiment, the invention provides a kit containing one or more components of the liposomes or formulations of the invention and instructions on how to combine and use the components and the formulation resulting from the combination. In various embodiments, the kit includes a solution of the sparingly water-soluble agent in an aprotic solvent in one vial and a liposome preparation containing the components to form a transmembrane gradient in another vial. In various embodiments, the kit includes a solution of the chelating agent with the membrane transfer enhancer and a liposome preparation containing the components to form a transmembrane gradient in another vial. Also included are instructions for combining the contents of the vials to produce a liposome encapsulated chelating agent or a formulation thereof of the invention. In various embodiments, the amount of chelating agent and liposome are sufficient to formulate a unit dosage formulation of the encapsulated chelating agent. In the context of iron removal in a patient suffering from iron transfusional overload diseases, one unit of a unit dosage formulation of the liposome chelating agent of the invention is sufficient to chelate at least about 220 mg of iron in a patient.

In an exemplary embodiment, the invention provides a kit for preparing a chelating agent liposome of the invention. An exemplary kit includes one vial containing a liposome or liposome solution, which is used to convert a premeasured amount of a lyophilized chelating agent (also included in the kit) into a liquid formulation of the liposome encapsulated chelating agent, e.g., at the bedside for administration into a patient. In an exemplary embodiment, the contents of the vials are sufficient to formulate a unit dosage formulation of the chelating agent.

The following non-limiting Examples are offered to illustrate selected embodiments of the invention.

### EXAMPLES

### Example 1

### General Liposome Preparation.

Prior to liposome formation, lipids are dissolved in chloroform, and chloroform is removed under reduced pressure using a rotary evaporator to form a thin lipid film on the sides of a glass flask. The lipid film is dried overnight under a high vacuum. The lipid film is rehydrated with a 250 mM solution of ammonium sulfate (ammonium sulfate buffer). The preparations of liposomes that are described in Example 1 are given below but the method is applicable to every formulation mentioned. The liposomes were composed of either DOPC/Cholesterol or HSPC/Cholesterol with varying ratios i.e. 3/0, 3/0.5, 3/1, 3/1.5 3/2. Lipids in the solid form were weighed out in the required amounts and dissolved in ethanol at a concentration of 500 mM phospholipid at 65° C. Ammonium sulfate solution was prepared by dissolving solid ammonium sulfate (Spectrum Chemicals A1245 Lot# YL0780) in deionized water to a final concentration of 250 mM. 9 volumes of pre-warmed (65° C.) ammonium sulfate solution was added and the mixture was mixed well. It was transferred to a 10 ml thermostatically controlled Lipex Extruder. The extruder temperature was held at 25° C. for DOPC liposomes and 65° C. for HSPC liposomes. The liposomes were formed by extruding 10 times through polycarbonate membranes having 0.1 um pores. After extrusion the liposomes were cooled on ice. The transmembrane electrochemical gradient was formed by purification of the liposomes by dialysis in dialysis tubing having a molecular weight cut off of 12,000-14,000. The samples are dialyzed against 5 mM HEPES, 10% sucrose pH 6.5 (stirring at 4° C.) at volume that is 100 fold greater than the sample volume. The dialysate was changed after 2 h then 4 more times after 12 h each. The conductivity of the liposome solution was measured and was indistinguishable from the dialysis medium ˜400/cm.

In the case of liposomes with diameters less than 350 nm they are filtered through a 0.45 micron sterile filter into a sterile container. Multilamellar (MLV) or oligolameller (OLV) vesicles are prepared under aseptic conditions using pre-sterilized buffers (Liposome Technology: Liposome preparation and related Techniques, 3^{rd} addition, 2006, G. Gregoriadis, ed.). Following their manufacture in ammonium sulfate buffer and dialysis against 100 volumes of sucrose buffer they are extruded through a 2 micron polycarbonate membrane into a sterile container. The usually total lipid concentration before dialysis of LUV is 20 mM and of MLV is 100 mM, unless otherwise indicated. Average liposome diameter and zeta potential are determined by dynamic light scattering measurements (Malvern Instruments Zetasizer Nano ZS). For liposomes extruded through the 100 nm polycarbonate membrane the liposome diameter is approximately 100 nm. For LMV, MLV or OLV liposomes the diameters can range from 0.5 microns to 40 microns before extrusion after 0.5 to 3 microns extrusion through the 2 micron polycarbonate membrane depending upon the preparation.

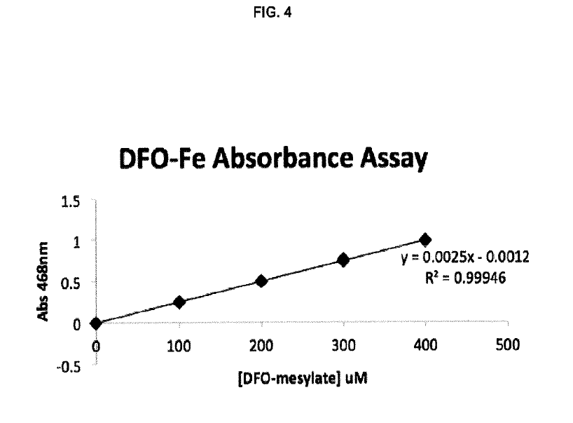

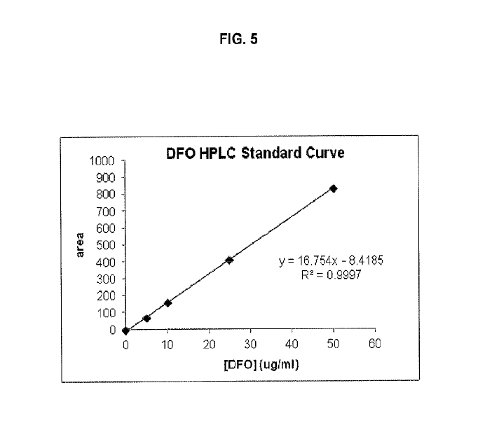

Deferoxamine (DFO) Quantification. Liposome encapsulated DFO was quantified by a DFO assay that utilizes the high absorption of DFO-Fe complex at 468 nm after liposome disruption by Triton X-100 (Lau E H, et al. Br J Haematol. 1981 April; 47(4):505-18) (standard curve is in FIG. 4) or by HPLC. HPLC analysis of DFO was performed on HPLC using an Agilent 1100 HPLC with and Agilent Zorbax 5 um, 4.6×150 mM, Eclipse XDB-C8 column. The mobile phase consists of A=0.1% TFA, B=0.1% TFA/Acetonitrile with a gradient elution starting at 5% B and increasing to 22% B in 12 min with 5 min equilibration back to 5% B. The flow rate is 1.0 ml/min, column temperature is 30° C., 10 ul injection and detection by absorbance at 220 nm. The retention time of DFO is 9.7 min. The standard curve for DFO quantification is in FIG. 4. The standard curve for the DFO HPLC assay is in FIG. 5.

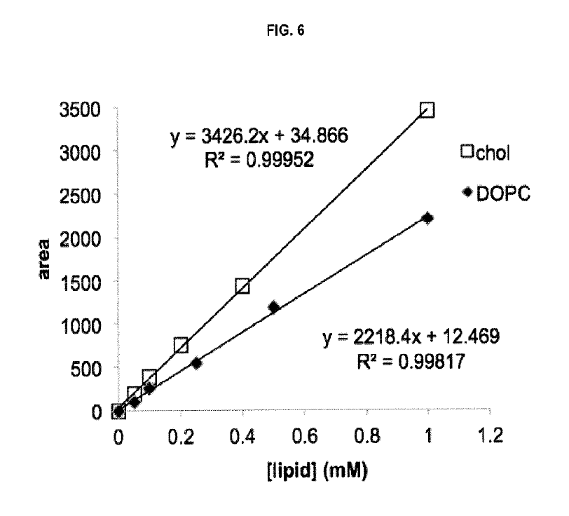

The lipid components of the liposomes were quantified using by HPLC using an Agilent 1100 HPLC with and Agilent Zorbax 5 um, 4.6×150 mM, Eclipse XDB-C8 column and a mobile phase of A=0.1% TFA, B=0.1% TFA/MeOH with an isocratic elution of 99% B. The flow rate is 1.0 ml/min, column temperature is 50° C., 10 ul injection and detection by absorbance at 205 nm. The retention times for lipids used are as follows: cholesterol 4.5 min, DOPC 6.2, POPC 6.4. The standard curve for the lipids is shown in FIG. 6. A phosphate assay was also used for phospholipid concentration determination (Bartlett G R, J. Biol. Chem. 234,466 (1959)). The liposome size is measured by dynamic light scattering.

Liposome Loading Desferoxamine mesylate (DFO) (Sigma, D9533, lot#SLBB5561V) was dissolved in deionized water at a concentration of 50 mg/ml. The DFO was introduced to the liposomes at a D/L ratio of 150 g drug/mol phospholipid (drug to total lipid ratio (wt/wt) of 0.176). The liposomes were diluted with 50 mM 3-(cyclohexylamino)-1-propanesulfonic acid (Sigma C2632) (CAPS), 10% sucrose pH 9 to a final volume of 1 mL. Varying volumes of ethanol (Gold Shield, 200 proof, Hayward, Calif.) were added and the final % ethanol described as the % added i.e. 0.2 mL ethanol added to 1 mL aqueous solution is 20% v/v. HSPC samples were heated at 65° C. and DOPC samples at 37° C. for 1 h. After heating all samples were placed on ice for 15 min. The liposomes were vortexed and 100 uL of sample was kept as the “before column” and the rest purified on a Sephadex G25 column (equilibrated with Hepes buffered saline, pH 6.5, HBS). The turbid fraction (liposomes) was collected and analyzed for drug and lipid as described in Experimental Methods. The degree of liposome DFO loading is quantified by measuring the drug and lipid after loading and purification and comparing to the input drug and lipid ratio (D/L). % Loading Efficiency=(D/L) purified/(D/L) input×100. The loading capacity of the liposomes is quoted as micrograms drug per micromole phospholipid (or g/mol).

### Example 2

Remote Loading of DFO into Preformed Liposomes.

Liposomes were prepared and purified as described above in Example 1. After loading and purification by gel filtration chromatography (Sephadex G25 column equilibrated with HBS, pH 6.5) an aliquot of each sample was dissolved in methanol and analyzed by HPLC using methods for both DFO and Chol detection described in Example 1. The results are listed in Table 1. DFO was undetected in any of the samples (Table 1). As the loading technique used here was similar to many published reports of remote loading drugs with titratable amines, this means that DFO does not remote load under the previously described remote loading conditions. The absence of loading observed was attributed to the high water solubility of DFO and its complicated solution ionization properties (Ihnat et al. 2000 89, 1525-1536).

TABLE 1
Remote loading DFO into preformed liposomes containing ammonium sulfate and zinc sulfate.

| | | input D/L | | time | temp | tonic | % loading |
|---|---|---|---|---|---|---|---|
| lipid (molar ratio) | loading agent | (g/mol) | pH | (h) | (C.) | agent | efficiency |
| 100 DOPC/67 Chol/1 PEG-DSG | 90 mM ZnSO4, 60 mM (NH4)2SO4 | 400 | 6.5 | 0.5 | RT | 10% sucrose | 0 |
| 100 DOPC/67 Chol/1 PEG-DSG | 90 mM ZnSO4, 60 mM (NH4)2SO4 | 400 | 6.5 | 0.5 | RT | 10% sucrose | 0 |
| 100 DOPC/67 Chol/1 PEG-DSG | 180 mM ZnSO4, 120 mM (NH4)2SO4 | 400 | 6.5 | 0.5 | 65 | 10% sucrose | 0 |
| 100 HSPC/67 Chol/1 PEG-DSG | 250 mM (NH4)2SO4 | 400 | 6.5 | 0.5 | 65 | 10% sucrose | 0 |
| 100 HSPC/67 Chol/1 PEG-DSG | 250 mM (NH4)2SO4 | 400 | 7.5 | 0.5 | 65 | 10% sucrose | 0 |
| 100 DOPC/67 Chol/1 PEG-DSG | 250 mM (NH4)2SO4 | 400 | 7.5 | 0.5 | 65 | 10% sucrose | 0 |
| 100 DOPC/67 Chol/1 PEG-DSG | 250 mM (NH4)2SO4 | 400 | 6.5 | 0.5 | 65 | 10% sucrose | 0 |
| 100 DOPC/67 Chol/1 PEG-DSG | 90 mM ZnSO4, 60 mM (NH4)2SO4 | 400 | 7.5 | 0.5 | RT | 10% sucrose | 0 |
| 100 HSPC/67 Chol/1 PEG-DSG | 250 mM (NH4)2SO4 | 400 | 5 | 0.5 | 70 | 10% sucrose | 0 |
| 100 HSPC/67 Chol/1 PEG-DSG | 250 mM (NH4)2SO4 | 400 | 8 | 0.5 | 70 | 10% sucrose | 0 |
| 100 HSPC/67 Chol/1 PEG-DSG | 250 mM (NH4)2SO4 | 400 | 9 | 0.5 | 70 | 10% sucrose | 0 |
| 100 DOPC/67 Chol/1 PEG-DSG | 90 mM ZnSO4, 60 mM (NH4)2SO4 | 400 | 5 | 0.5 | 70 | 10% sucrose | 0 |
| 100 DOPC/67 Chol/1 PEG-DSG | 90 mM ZnSO4, 60 mM (NH4)2SO4 | 400 | 9 | 0.5 | 70 | 10% sucrose | 0 |

Table 1. HSPC and DOPC containing liposomes were prepared as described above and DFO added to the solution. The samples were incubated at various pH's and temperatures and after loading process was quenched by cooling on ice the samples were purified from any unloaded DFO the DFO content of the liposomes was measured.

### Example 3

DFO can be Remote Loaded if a Membrane Transfer Enhancer is Added to the DFO-Liposome Mixture.

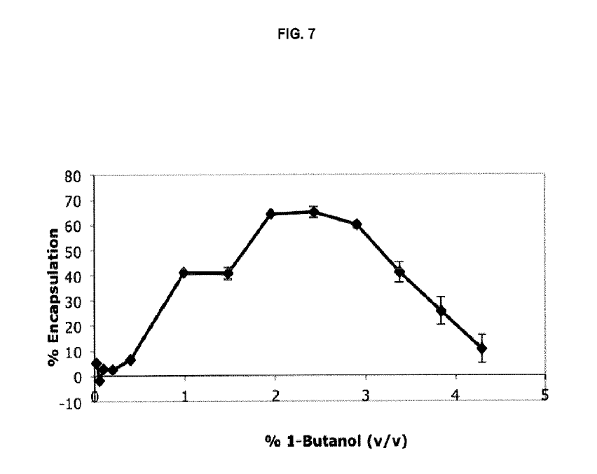

Liposomes were prepared and purified as described previously in Example 1. DFO was added to purified liposomes at 200 g/mol. After pH adjustment to 8, 1-butanol was added slowly while vortexing in amounts corresponding to 0.1, 0.25, 0.5, 1, 2, 5, 10 and 20% v/v. In the absence of 1-butanol no DFO loading was observed. Remarkably, at concentrations greater than 0.5% (v/v) loading efficiencies increase and reach almost 65% at 2% (v/v) (FIG. 7). Increasing the 1-butanol content beyond 3% dramatically reduces the efficiency of loading. The presence of butanol as a membrane transfer reagent is required for remote loading of DFO into ammonium sulfate containing liposomes.

### Example 4

Ethanol Also Functions as a Membrane Transfer Enhancer to Enable the Remote Loading of DFO into Liposomes.

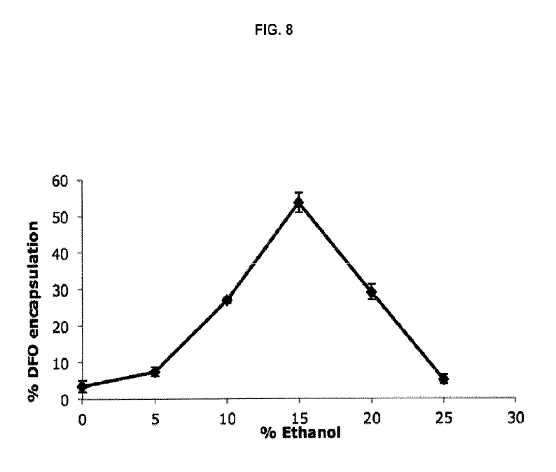

Liposomes were prepared and purified as described in Experimental Methods. DFO was added to purified liposomes at 200 g/mol. After pH adjustment to 8, ethanol was added slowly while vortexing in amounts corresponding to 0, 5, 10, 15, 20 and 25% v/v. Samples were incubated for 20 min at 45° C. to initiate loading, after which time they were chilled and purified as described in Experimental Methods. The final DFO/lipid ratio was calculated after drug and lipid was analyzed as described in Example 1. In the absence of ethanol, no DFO loading was observed (FIG. 8). At ethanol concentrations greater than 5% (v/v) loading efficiencies increase and reach 55% at 15% (v/v). Increasing the ethanol content beyond 15% dramatically reduces the efficiency of loading. The presence of a membrane transfer enhancer is required for remote loading of DFO into ammonium sulfate containing liposomes made of POPC and Chol.

### Example 5

An Electrochemical Gradient is Required to Observe Loading of DFO into Preformed Liposomes.

Liposomes were extruded through 0.1 nm polycarbonate membranes in either 5 mM Hepes, 10% (w/w) sucrose, pH 6.5 or ammonium sulfate. Samples were purified as described before. The loading conditions were identical and DFO and lipid were analyzed as described in Example 1. The liposome sample containing sucrose showed no ability to internalize DFO but the ammonium sulfate liposome internalized about 53% of the available drug to yield a liposome containing 174.9 ug/umol DFO (see Table 2). Thus, the presence of an electrochemical is required to remote load DFO inside a liposome. In the presence of a membrane transfer enhancer, in this case 20% (v/v) ethanol, DFO loading is efficient but it does not occur unless both the gradient and the membrane transfer enhancer are present during loading.

TABLE 2
The requirement of an electrochemical gradient to
facilitate loading of DFO into preformed liposomes.

| | | Final | | |
|---|---|---|---|---|
| Lipid | Internal | [Ethanol] | Input D/L | Output D/L |
| Formulation | Solution | v/v | (g/mol) | (g/mol) |
| DOPC/Chol | Sucrose 10% | 20% | 200 | −0.5 ± 0.6 |
| (3/0.5) | (w/w) | | | |
| DOPC/Chol | (NH4)₂SO₄ | 20% | 200 | 52.4 ± 0.4 |
| (3/0.5) | 250 mM | | | |

Table 2. Loading efficiency of DFO into liposomes of identical lipid composition but varying in the composition of the solution on the liposome interior.

### Example 6

Effect of Incubation Time at 37 C on the Loading Efficiency of DFO into Liposomes Composed of DOPC.

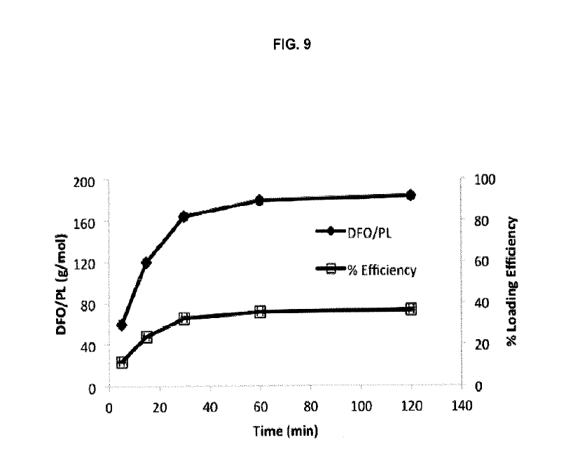

DOPC/Chol (3/0.5 mol/mol) liposomes containing 250 mM ammonium sulfate were prepared and purified as described in Example 1. The solution pH was adjusted to 9 and divided into multiple eppendorf tubes and incubated at 37° C. At designated time points samples was removed and put on ice and then purified. Drug and lipid measurements were performed and the results plotted above. The input ratio was 500 g/mol DFO to lipid and 20% ethanol was used. The results in FIG. 9 indicate that remote loading is aided by incubation for at least 30 min (45 min is optimal in these conditions) after which very little change takes place in the loading efficiency up to 2 h. At pH 9, incubation for at least 30 min is recommended for optimal DFO liposome loading.

### Example 7

The Effect of Temperature on Loading Efficiency of DFO into Ammonium Sulfate Containing Liposomes.

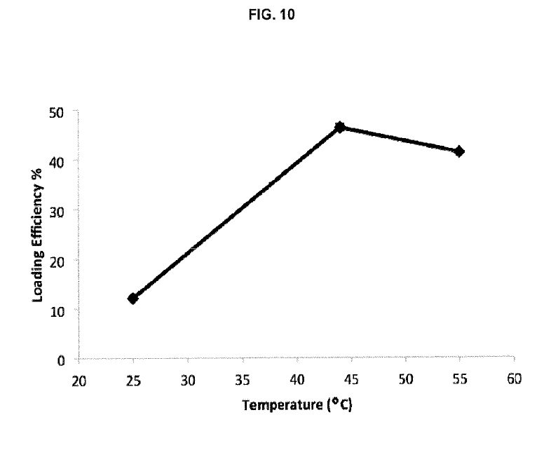

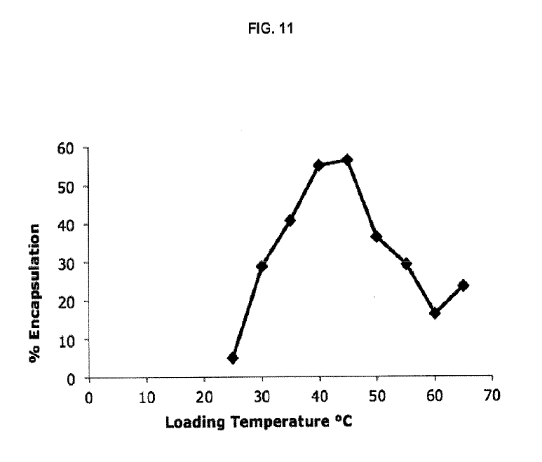

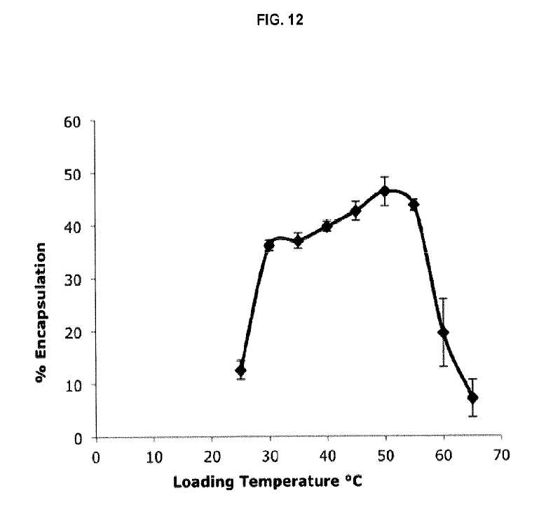

Liposomes formed from DOPC/Chol (3/0.5 mol/mol), POPC/Chol/DSPG (3/0.5/0.15 mol/mol/mol) liposomes or POPC/Chol (3/0.5 mol/mol) containing 250 mM ammonium sulfate were prepared and purified as described in Example 1. The initial D/L ratio was 500 g/mol and the samples were heated for 1 h with 20% ethanol added. The three liposome formulations displayed differing sensitivity to changes in temperature in regard to loading efficiency. Of the temperatures studied, 45° C. had the highest loading efficiency for DOPC/Chol (FIG. 10) and POPC/Chol/DSPG (FIG. 11) liposomes while for POPC/Chol liposomes (FIG. 12) 50° C. was slightly better. In all cases higher temperatures decreased the loading efficiency, possibly as a result of liposome destabilization in the presence of 20% ethanol. Incubating the samples at 45° C. provided the best loading of DFO using these liposomes in the presence of 20% ethanol for the compositions tested.

### Example 8

The Effect of Solution pH on Loading Efficiency of DFO into DOPC Containing Liposomes.

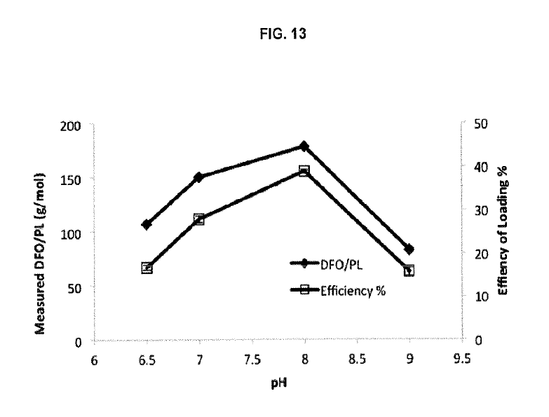

Liposomes composed of DOPC/Chol (3/0.5 mol/mol) containing 250 mM ammonium sulfate were prepared and purified as described in Example 1 except the dialysis media contained no buffer. After dialysis, the liposomes were divided into two aliquots and either Hepes buffer was added for pH<8 or CAPS buffer was added for pH>8. The initial D/L ratio was 500 g/mol and the samples were heated for 1 h with 20% ethanol added. Of the pH conditions studied, the pH for achieving highest DFO remote loading was 8 (FIG. 13).

### Example 9

Using Optimized pH, Incubation Time and Temperature to Load DFO into DOPC Liposomes at Various Input DFO to Lipid Ratios.

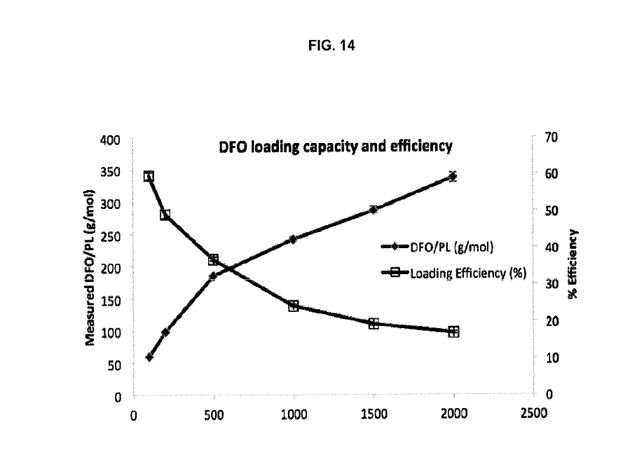

Liposomes were prepared containing 250 mM ammonium sulfate and purified as described in Example 1. Varying amounts of DFO was added to a constant amount of liposomes to adjust the input DFO to phospholipid ratio. The pH was adjusted to 8 and samples were heated at 45° C. for 45 min. The efficiency reaches 60% at 100 g/mol but progressively lowers as the input D/L increases. The highest D/L tested, 2000 g/mol had an efficiency of 17% and a capacity of 337 g/mol (FIG. 14). Using the present techniques, high DFO-to-lipid rations can be achieved by remote loading, although at the expense of the loading efficiency.

### Example 10

Using Optimized pH, Incubation Time and Temperature to Load DFO in DOPC Liposomes Containing 500 mM Sulfate as a Function of the Input DFO to Lipid Ratio.

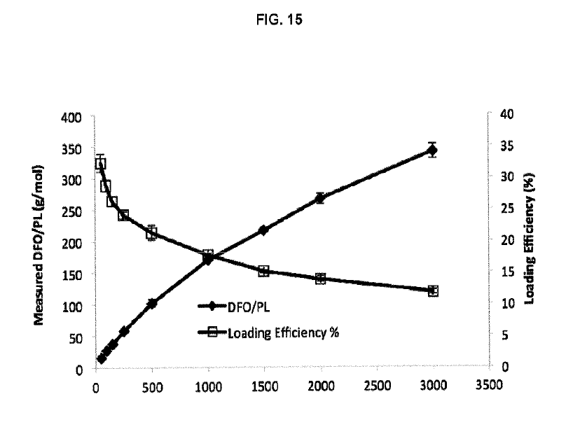

Liposomes were made using 500 mM ammonium sulfate and purified as above. Varying amounts of DFO was added to a constant amount of liposomes to vary the input DFO to phospholipid ratio, with additional sucrose added to balance tonicity. The pH was adjusted to 8 and samples were heated at 45° C. for 45 min. The loading efficiency is dependent on the input D/L ratio (FIG. 15). At 50 g/mol DFO to lipid the efficiency reaches 32% and at 100 g/mol is 29%. However, as observed in Example 9 above (with 250 mM ammonium sulfate), the higher the input D/L the higher the resultant D/L but the lower the efficiency. The highest loading ratio achieved was 341 g/mol. High DFO-to-lipid rations can be achieved by remote loading.

### Example 11

The Influence of Internal Sulfate Concentration the Amount of DFO Encapsulated in the Liposome.

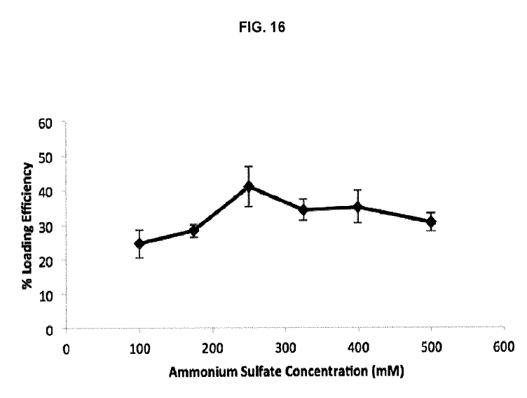

Liposomes were made using ammonium sulfate solutions of varying concentrations. After purification, DFO was added at a constant DFO to phospholipid ratio 100 g/mol. The pH was adjusted to 8 and ethanol added (15% v/v) and samples were heated at 45° C. for 45 min. The loading efficiency is dependent on the intraliposomal [SO₄]. The highest efficiency was achieved at 250 mM. (FIG. 16). The highest loading efficiency was achieved using 250 mM ammonium sulfate. Higher internal concentrations had reduced loading efficiencies.

### Example 12

Remote Loading DFO into DOPC Liposomes Containing Ammonium Diethylenetriamine Pentaacetate (DTPA) with Varying Chol Content as a Function of Ethanol Content.

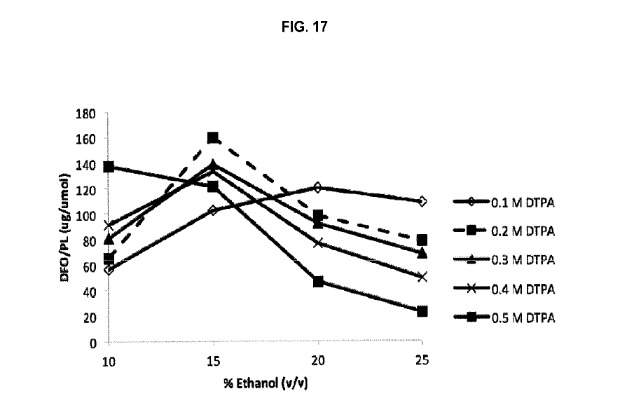

The acid form of DTPA (Spectrum Labs D2323) was titrated to pH 6.4 with ammonium hydroxide and liposomes were prepared and purified as above using this as the aqueous solution. The liposomes were loaded as described in Example 1 except the loading conditions were that the samples were incubated at pH 8 for 1 h min at 45° C. DFO could be remote loaded using NH4-DTPA as a trapping agent. The highest concentration of intraliposomal DTPA used (500 mM carboxylate equivalents) required 10% ethanol for optimal loading while the lowest concentration tested required 20% (FIG. 17). The intermediate DTPA concentrations were optimal at 15% ethanol. This is an example of an FDA approved chelating agent being used to remote load the iron chelating agent DFO into a liposome.

### Example 13

Remote Loading Efficiency DFO into DOPC Liposomes Containing Triethylamine Dextran Sulfate (TEA-DS).

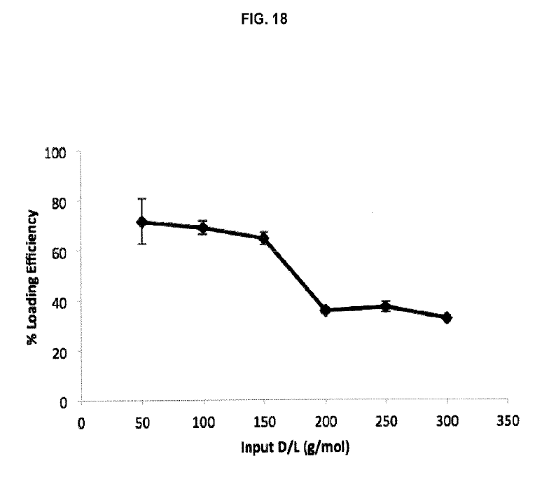

Triethylammonium dextran sulfate was prepared using Dowex 50Wx8-200 ion exchange (changed with HCl) resin to acidify the dextran sulfate which was then titrated with triethylamine to a pH in the range of 6.8-8.0. The solution was then diluted with water to a concentration of 0.5M sulfate equivalents. Liposomes were prepared as in Example 1 except using TEA dextran sulfate instead of ammonium sulfate and were purified by anion exchange (Amberlite IRA-67) and dialysis prior to loading with DFO. Liposomes containing TEA dextran sulfate are able to enable remote loading of DFO (FIG. 18) and display a similar behavior in terms of the loading efficiency as the (NH₄)₂SO₄ containing liposomes described in Example 10 (FIG. 16.) Under these conditions, TEA dextran sulfate is equally capable of loading DFO as ammonium sulfate.

### Example 14

Remote Loading DFO by Remote Loading into Liposomes Containing Ammonium Sulfate and Zinc Sulfate Ions.

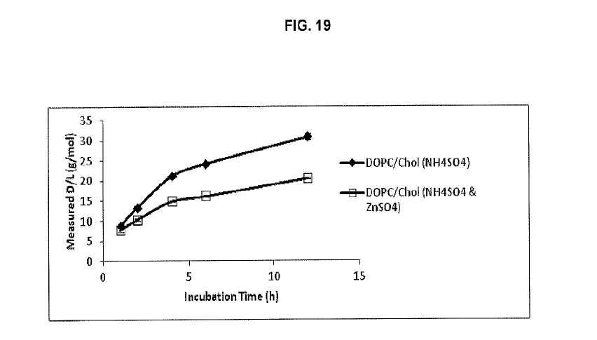

Liposomes prepared as in Example 1, are incubated with DFO (at 150 g/mol) in 15% ethanol at 37° C. for various amounts of time. At the indicated times, liposomes were removed, purified and the resultant DFO and phospholipid content measured. Transmembrane electrochemical gradients that have a metal component are of interest as they may allow for enhanced chelating agent retention within the liposome. Even more important is that chelating agents such as DFO can remove other therapeutically important endogenous metals such as zinc. Remote loading occurred when some of the ammonium sulfate was replaced with zinc sulfate (FIG. 19).

### Example 15

Remote Loading DFO Using a Calcium Acetate Gradient.

Liposomes were prepared with a calcium acetate internal solution as described in Example 1. DFO was added at 150 g/mol and the final buffer composition was 50 mM Hepes, 10% sucrose. The sample was divided into 4 aliquots and the pH adjusted to 6.9, 8.1, 8.9 and 9.8 for each of the aliquots. 20% (v/v) ethanol was added and the samples were heated to 37° C. for 30 min. After purification, the drug and lipid was quantified and is shown in Table 3. Remote loading of DFO was not achievable using the acetate gradient technique at any of the pH tested.

TABLE 3
Results from attempts to load DFO using an acetate loading
technique.

| Liposome | Internal | | |
|---|---|---|---|
| Formulation | Trapping Agent | Loading pH | Output DFO/PL |
| POPC/Chol (3/0.5) | 0.12M calcium | 6.93 | DFO Undetectable |
| | acetate | | |
| POPC/Chol (3/0.5) | 0.12M calcium | 8.14 | DFO Undetectable |
| | acetate | | |
| POPC/Chol (3/0.5) | 0.12M calcium | 8.9 | DFO Undetectable |
| | acetate | | |
| POPC/Chol (3/0.5) | 0.12M calcium | 9.8 | DFO Undetectable |
| | acetate | | |

### Example 16

Comparison of Passive Loading to Remote Loading of Deferoxamine in Liposomes.

The DFO was ‘passively loaded’ into liposomes composed of DOPC/Cholesterol (3 mol/0.5 mol) and extruded as described in Example 1. The aqueous portion of the extruding solution consisted of 300 mg/ml desferoxamine methanesulfonic acid. After extrusion the unencapsulated DFO was removed using a Sephadex G25 size exclusion column. The resulting DFO to lipid ratio was 84.8 g drug/mol DOPC. The passively encapsulated liposomal DFO was sterile filtered and placed in storage at 4° C. Remote loaded liposomal DFO was composed of DOPC/Cholesterol (3 mol/0.5 mol) and extruded as described in Example 1 (100 nm membrane pores). 140 mg/mL ammonium sulfate was the trapping agent and the loading was performed by incubating for 60 min at 37° C. and pH 9. After extrusion the unencapsulated DFO was removed using a Sephadex G25 size exclusion column. The comparison of the loading efficiency and drug to lipid ratio between the two methods is shown in Table 4. The remote loaded liposomes were able to achieve a higher drug to lipid ratio while also achieving a higher loading efficiency than passive loading.

TABLE 4
Liposome formulations of DFO were prepared by either rremote
loading or passive loading.

| | | Encapsulated | Loading |
|---|---|---|---|
| | Drug loading ratio | drug ratio | efficiency |
| formulation | (g drug/mol DOPC) | (g drug/mol DOPC) | (%) |
| Passive | 4000 | 234 ± 1.66 | 5.9 |
| encapsulated | | | |
| liposomal DFO | | | |
| Remote loaded | 1115 | 248 ± 8.77 | 22.5 |
| liposomal DFO | | | |
| Remote loaded | 3346 | 361 ± 3.52 | 10.8 |
| liposomal DFO | | | |

### Example 17

Storage Stability of Liposomal Desferoxamine at 4° C.

The Passively loaded liposomal DFO was composed of DOPC/Cholesterol (3 mol/0.5 mol) and extruded as described in Experimental Methods. The aqueous portion of the extruding solution consisted of 100 mg/ml deferoxamine methanesulfonic acid. After extrusion the unencapsulated DFO was removed using a Sephadex G25 size exclusion column. The resulting DFO to lipid ratio was 84.8 g drug/mol DOPC. The passively encapsulated liposomal DFO was sterile filtered and placed in storage at 4° C. Remote loaded liposomal DFO was composed of DOPC/Cholesterol (3 mol/0.5 mol) and extruded as described in Experimental Methods (100 nm membrane pores). 250 mM ammonium sulfate was the trapping agent and the loading was performed by incubating for 45 min at 37° C. and pH 8. After extrusion the unencapsulated DFO was removed using a Sephadex G25 size exclusion column. The resulting DFO to lipid ratio was 241.3±3.4 g drug/mol DOPC. The remote loaded liposomal DFO was sterile filtered and placed in storage at 4° C. The passively loaded and remote loaded liposomal DFO both showed good storage stability at 4° C. (Table 5). The remote leaded formulation contained 2.8-fold more DFO and retained the chelating agent as well as or better than the passive loaded liposome formulation.

TABLE 5
Liposome formulations at DFO were stored in solution at
4° C. tor 5 months and the drug retention in the
liposome is stated as %.

| formulation | Drug retention in liposome |
|---|---|
| Passive encapsulated | 94.7 ± 0.91% |
| liposomal DFO | |
| Remote loaded liposomal | 101.6 ± 2.17% |
| DFO | |

### Example 18

Effect of the Membrane Transfer Alcohol Type on Efficiency of Loading Liposomal with Deferoxamine.

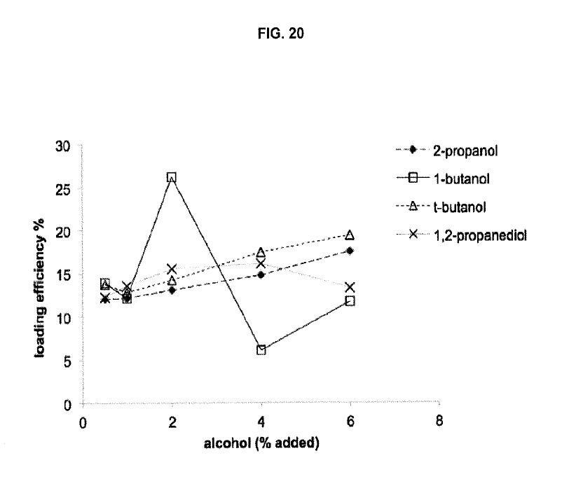

Liposomal composed of HSPC/Cholesterol (3 mol/2 mol) prepared in 250 mM ammonium sulfate and extruded at 65° C. through 100 nm membrane pores as described in Example 1 were loaded with DFO by incubating for 10 h at 65° C. and pH 9. The resulting DFO to lipid ratio was 241.3±3.4 g drug/mol HSPC (FIG. 20). The membrane transfer enhancer, 1,2-propanediol reaches a maximum alcohol content before 6% with a maximum loading efficiency of 15.5%. 2-propanol and t-butanol did not reach a maximum alcohol content before a total concentration of 6%. 1-butanol appears to increase loading efficiency at 2% but higher concentrations of 1-butanol disrupt the liposome.

### Example 19

Effect of the DFO Concentration on Efficiency of Loading Liposomes with DFO.

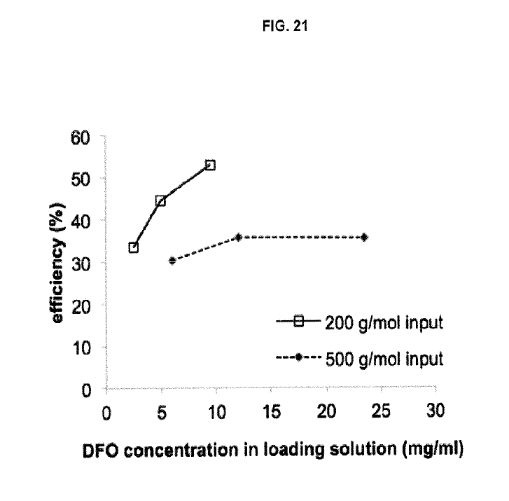

Liposomes composed of DOPC/Cholesterol (3 mol/0.5 mol), were prepared in 250 mM ammonium sulfate and extruded through polycarbonate membranes with 100 nm pores as described in Example 1, the liposomes were loaded at various DFO external concentrations by incubating for 30 min at 37° C. and pH 8.0 (FIG. 21). The unencapsulated DFO was removed using a Sephadex G25 size exclusion column. Using an input DFO to lipid ratio of 200 g drug/mol DOPC there is a dependence of the loading efficiency on the DFO concentration in the loading solution even when the drug to lipid ratio remains constant at 200 g drug/mol DOPC. This effect is not observed using an input drug to lipid ratio of 500 g drug/mol DOPC (FIG. 21).

### Example 20

The Effect of Time and Temperature on Active Loading of DFO into Liposomes in the Presence or Absence of a Chemical Membrane Modifier.

The Effect of Temperature on Loading Efficiency of DFO into Ammonium Sulfate.

The effect of temperature on active loading of DFO into liposomes was evaluated with and without the presence of a membrane modifier (ethanol). The temperature ranges of 0-100° C. which are defined as typical in Paragraph [0091] were tested in the presence and absence of the membrane modifier ethanol.

Liposomes actively loaded using ethanol as a membrane modifier were formed from POPC/Chol (3/0.5 mol/mol) containing 250 mM ammonium sulfate were prepared and purified as described in Example 1. The target drug to lipid ratio of 500 g DFO/mol PL was used. The loading solution contained a concentration of 20% ethanol as the membrane modifier and the active loading was accomplished by heating at the indicated temperature for 1 hour.

Active loading of DFO using no ethanol (or other membrane modifier) was done using liposomes composed of either a fluid-phase lipid (egg PC) or a gel-phase lipid (HSPC), specifically, egg PC liposomes (3:2 Chol, 0.5 M ammonium sulfate, 90 nm) or HSPC liposomes (3:2 Chol, 250 mM ammonium sulfate, 90 nm) were remote loaded with DFO at a temperature range of 40-120° C. (pH 8.0) for either 10 min or 30 min. Temperatures above 100° C. were obtained by placing the samples in sealed tubes so that the pressure in the tube increased when the samples were heated but the fluid did not boil. The target drug to lipid ratio was 170 g DFO/mol PL. After the DFO was loaded the tubes were rapidly cooled to room temperature. Unencapsulated DFO was removed by dialysis at 4° C., and drug/PL concentrations were analyzed by HPLC as described herein.

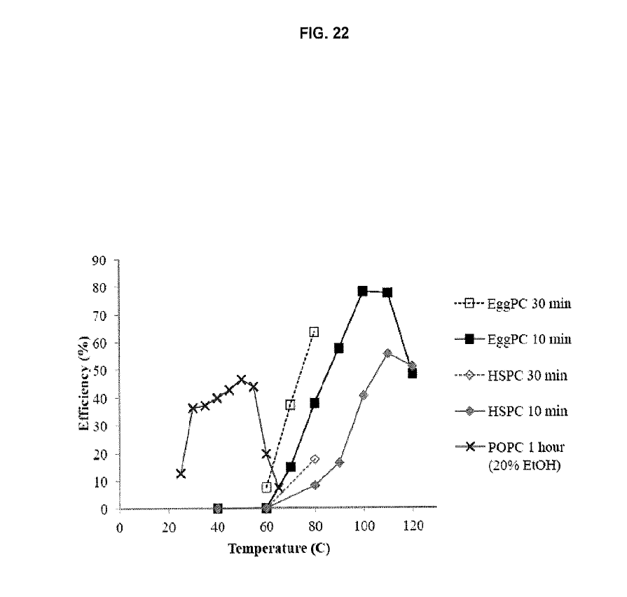

The temperature at which the maximum efficiency of DFO loading using 20% ethanol for a time of 1 hour was determined to be 50° C. (FIG. 22). The temperature at which the maximum efficiency for loading in the absence of ethanol at a time of 10 min was between 90-110° C. for both liposome compositions tested. In the absence of ethanol, the maximum efficiency was obtained at lower temperatures when the time was increased to 30 min but importantly no significant (>20% efficient) loading was observed below 60° C. in the absence of ethanol.

The active loading of DFO into liposomes can be accomplished without the presence of a chemical membrane modifier in the temperature range of 60-110° C. The active loading of DFO into liposomes is highly dependent on temperature for procedures that include a membrane modifier (ethanol) and also for procedures that do not include a membrane modifier. Higher temperature is required when no membrane modifier is present.

### Example 21

The Effect of Temperature on Active Loading of DFO and Doxorubicin.

The unique effect of temperature on active loading of DFO into liposomes as compared to other commonly used drugs was demonstrated by comparison of the loading of DFO with the loading of doxorubicin into liposomes of the same composition. This experiment was done in the absence of a chemical modifier. Liposomes were composed of either a fluid-phase lipid (egg PC) or a gel-phase lipid (HSPC), specifically, egg PC liposomes (3:2 Chol, 0.5 M ammonium sulfate, 90 nm) or HSPC liposomes (3:2 Chol, 0.25 M ammonium sulfate, 90 nm) were remote loaded with DFO or doxorubicin at a temperature range between 40-120° C. for 10 min. Doxorubicin concentration in the loading solution was 5 mg/mL and a target drug to lipid ratio of 170 g DFO/mol PL. DFO was loaded at pH 8 while doxorubicin was loaded at pH 6.5. Unencapsulated drug was removed by dialysis at 4° C., and drug/PL concentrations were analyzed by HPLC as described herein.

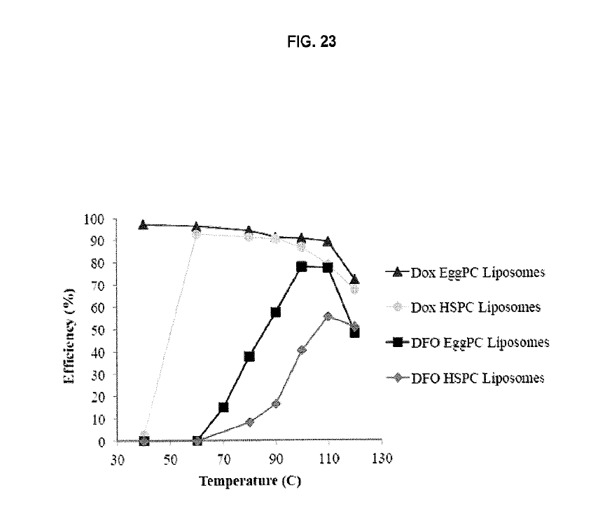

Liposomes composed of HSPC were actively loaded with doxorubicin at high efficiency (>90%) at temperatures slightly exceeding the phase transition temperature of HSPC (ie, 60° C. and above), but loaded very poorly below the lipid's phase transition temperature (ie, 40° C.) (FIG. 23) In contrast to the active loading of doxorubicin, DFO loaded poorly at temperatures exceeding the phase transition temperature for HSPC (60° C.), rather loading of DFO was efficient at temperature of >than 65° C. Loading of DFO reached a maximum efficiency at 100° C. Egg PC has a phase transition temperature below room temperature. Loading of DFO into liposomes composed of Egg PC only occurred at temperatures>than 65° C.; these temperatures are well above the phase transition temperature of Egg PC. Liposomes composed egg PC, were actively loaded with doxorubicin at high efficiency at all of the temperatures in the range of 30-110° C. However doxorubicin loading decreased when temperatures were greater than 100° C. In contrast to doxorubicin, active loading of DFO in egg PC liposomes required much higher temperatures, the doxorubicin loading at temperatures>100° C. DFO loading efficiency into HSPC liposomes was retained. Using a target loading ratio of 170 g DFO/mol PL, DFO is loaded well at 70-130° C. and reached a maximum of 80% efficiency at temperatures of 90-110° C.

Active liposome loading of DFO was highly dependent on high temperature, not phospholipid phase transition temperature, while active loading of doxorubicin into liposomes was highly dependent on the phase transition temperature of the phospholipid. Active loading conditions for doxorubicin are representative of other small molecule drugs where the optimum efficiency is reached within 2-10° C. of the phase transition temperature of the liposome membrane components. DFO is a rare exception that requires temperatures well above (at least 20° C. above) the phase transition temperature of the liposome components for efficient active loading.

### Example 22

Deferasirox Quantification.

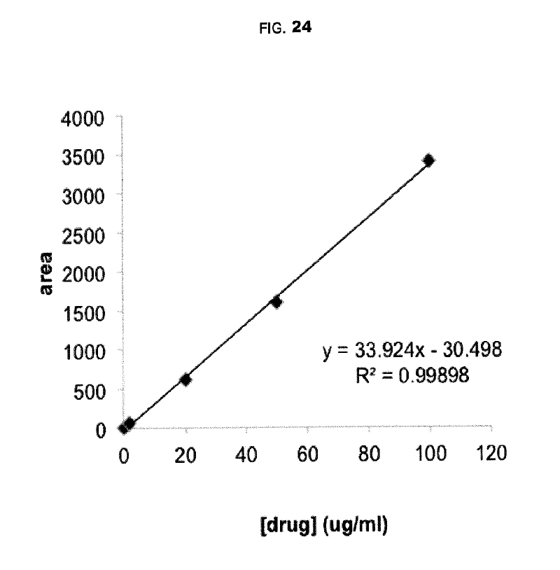

The HPLC analysis of deferasirox (Selleck Chemicals) was performed on the same system as described for Lipid Quantification in Example 1. The mobile phase consists of A=0.1% TFA, B=0.1% TFA/MeOH with a gradient elution starting at 50% B and increasing to 83% B in 13 min with 7 min equilibration back to 50% B. The flow rate is 1.0 ml/min, column temperature is 30 C, 10 ul injection and detection by absorbance at 254 nm. The standard curve is illustrated in FIG. 24. The retention time of deferasirox is 5.2 min.

### Example 23

Entrapment of Deferasirox in Liposomes that Contain a Calcium Acetate Gradient.

Calcium acetate solution was prepared by dissolving calcium acetate solid to a final concentration of 120 mM no pH adjustment was made to yield a final pH of 7.2. A 250 mM sodium sulfate was used as a control trapping agent solution which does not form an acetate gradient.

The liposomes were composed of either POPC/Cholesterol (3 mol/0.5 mol) or HSPC/Cholesterol (3 mol/0.5 mol). Lipids were dissolved in ethanol at a concentration of 500 mM phospholipid using either HSPC (423 mg/ml total lipid) or POPC (412 mg/ml total lipid) at 65° C. and then 9 volumes of the trapping agent solution heated to 65° C. was added to the ethanol/lipid solution also at 65° C. The mixture was vortexed and transferred to a 10 ml thermostatically controlled Lipex Extruder. The extruder temperature was held at 25° C. for POPC liposomes and 65° C. for HSPC liposomes. The liposomes were formed by extruding 10 times, through polycarbonate membranes having 100 nm pores. After extrusion, the liposomes were cooled on ice. The transmembrane electrochemical gradient was formed by replacing the external buffer by dialysis against 5 mM HEPES, 10% sucrose pH 6.5 (stirring at 4° C.) at volume that is 100 fold greater than the sample volume. The dialysate was changed after 2 h then 4 more times after 12 h each. The conductivity of the liposome solution was measured and was indistinguishable from the dialysis medium ˜40 μS/cm. The liposome size is measured by dynamic light scattering.

Deferasirox was dissolved in DMSO at a concentration of 20 mg/ml. The deferasirox was introduced to the liposomes at a deferasirox to HSPC ratio of 100 g drug/mol HSPC (drug to total lipid ratio (wt/wt) of 0.12). The liposomes were diluted with 50 mM MES, 10% sucrose pH 4.5 to increase the volume to a point where after addition of the drug the final DMSO concentration is 2%. The deferasirox/DMSO was added to the diluted liposomes, which were mixed at room temperature then transferred to a 45° C. bath for POPC liposomes and a 65° C. bath for HSPC liposomes and swirled every 30 s for the first 3 min of a total heating time of 30 min. After heating for 30 min all samples were placed on ice for 15 min. The loaded liposomes were vortexed and 100 ul of sample was kept as the “before column” and the rest transferred to microcentrifuge tubes and spun at 10,000 RPM for 5 min. The supernatants were purified on a Sephadex G25 column collected and analyzed by HPLC.

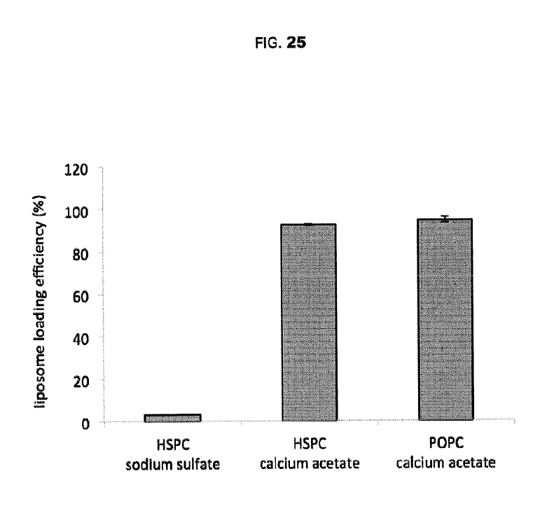

The loading of liposomes containing 250 mM sodium sulfate resulted in a loading efficiency of 3.3±0.14% for HSPC liposomes when the drug was added at 100 g drug/mol of HSPC lipid (FIG. 25). The loading efficiencies for liposomes containing calcium acetate were 92.5±0.33% (HSPC liposomes) and 94.8±1.46% (POPC liposomes). (FIG. 23). The unloaded deferasirox aggregates and forms a precipitate in the solution of sodium sulfate liposomes but no precipitate is seen in the suspension of liposomes containing calcium acetate. Liposomes of identical lipid matrix composition and size but varying in the composition of trapping agent had very different capabilities to load deferasirox. The liposome capable of generating an electrochemical gradient (calcium acetate) was able to load almost 100% of the drug at optimal conditions.

### Example 24

Effect of the Metal Counterion Used in the Acetate Gradient Formation for Remote Loading Deferasirox into Liposome.

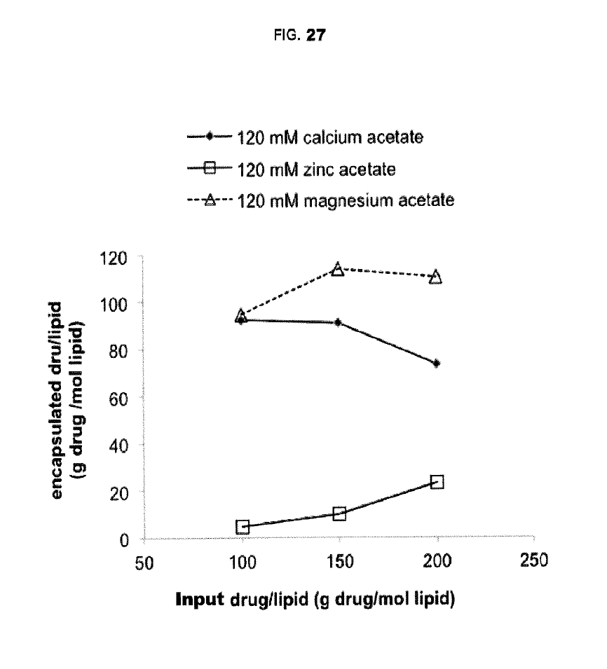

Zinc acetate and magnesium acetate solutions were prepared by dissolving each in water to a final concentration of 120 mM followed by adjustment of the pH to 4.2. The liposomes composed either POPC/Cholesterol (3 mol/0.5 mol) containing 120 mM of either calcium acetate, zinc acetate or magnesium acetate were prepared as described in Example 23. Deferasirox dissolved in DMSO was added to the liposomes at a ratio of 100, 150 and 200 g drug/mol POPC. Liposomes containing 120 mM zinc acetate gave the lowest encapsulation efficiency with a maximum drug to lipid ratio of 23.1±0.62 g drug/mol POPC (13.4% efficient). Liposomes containing 120 mM calcium acetate had a maximum drug from the input of 100 g drug/mol POPC, which resulted in 92.2±0.39 g drug/mol POPC (FIG. 27). Input ratios chelating agent/lipid higher than 100 had a lower chelating agent to lipid ratio in the final liposome. Liposomes containing 120 mM magnesium acetate also were loaded efficiently at an input of 100 g drug/mol POPC resulting in 94.6 g drug/mol POPC (FIG. 27). In addition liposomes loaded with the magnesium or zinc acetate gradients maintained the loading efficiency when the chelating agent/lipid ratio was greater than 150 g/mole (FIG. 27). Thus loading of deferasirox into liposomes is dependent on the metal counterion used in the acetate gradient formation. Although calcium and magnesium are similar, magnesium is the most effective at encapsulating deferasirox into liposomes.

### Example 25

Effect of Metal Acetate Concentration on Remote Loading Deferasirox into Liposomes.

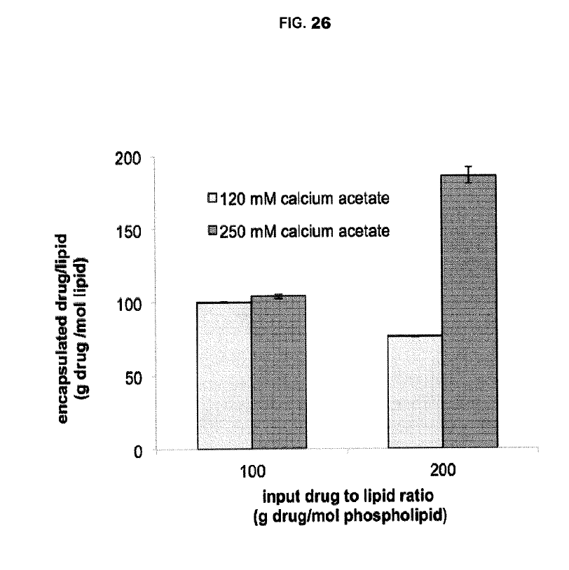

Liposomes composed of POPC/Cholesterol (3 mol/0.5 mol) containing either 120 mM or 250 mM calcium acetate were prepared as described in Example 23. Addition of deferasirox dissolved in DMSO to the liposomes at a ratio near 100 g drug/mol phospholipid results in a final encapsulated drug ratio of 100.2±0.41 g drug/mol phospholipid (99.6% efficiency) for liposomes containing 120 mM calcium acetate and 104.0±1.46 g drug/mol phospholipid (99.2% efficiency) for liposomes containing 250 mM calcium acetate. Addition of deferasirox to the liposomes at a ratio of 200 g drug/mol phospholipid results in a final encapsulated drug ratio of 76.4±0.29 g drug/mol phospholipid (40.7% efficiency) for liposomes containing 120 mM calcium acetate and 186.1±5.53 g drug/mol phospholipid (92.0% efficiency) for liposomes containing 250 mM calcium acetate. (FIG. 26). The liposome remote loading capacity for deferasirox is dependent upon the internal concentration of calcium acetate. An internal calcium acetate concentration of 250 mM enables efficient loading at 200 g drug/mol phospholipid (FIG. 26).

### Example 26

Loading of Etidronic Acid (1-Hydroxyethane 1,1-Dihydroxy Bisphosphonate), (EHBP) in Liposomes to be Added.

EHBP solution 60% w/w (Spectrum Labs E3490) was diluted to 0.3M (phosphate), the pH was adjusted to 7.2 with sodium hydroxide and was used as the aqueous dispersant prior to extruding liposomes. Liposome formulations consisting of DOPC/Chol/DSPG (3/2/0.15) and POPC/Chol/DSPG (3/2/0.15) were prepared. The corresponding lipids were weighed out and dissolved in hot ethanol at 65° C. Pre-heated EHBP (65° C.) solution was mixed with the ethanolic lipid solution forming multilamellar vesicles. The solutions were allowed to cool to room temperature and were extruded through 0.1 um polycarbonate membranes. The phospholipid concentration was 50 mM during the extrusion step. The resulting unilamellar liposomes were dialyzed exhaustively against 5 mM Hepes, 10% (w/w) sucrose, pH 6.5 at 4° C. The resultant osmolality and zeta potential of the DOPC/Chol/DSPG sample and POPC/Chol/DSPG sample was 331 mOsm/kg and -6.97 mV and 333 mOsm/kg and -13.4 mV respectively. The liposome formulations were also lyophilized and reconstituted with sterile water and the sizes before and after lyophilization given below as measured by dynamic light scattering. The Z-average size of the DOPC liposomes was 113.6 nm and 117.4 nm pre and post lyophilization and for the POPC liposomes 119.6 nm and 107.4 nm respectively. The reconstituted liposomes had at most a 10% change in size after reconstitution.

### Example 27

Remote loading of DFO or deferasirox into liposomes containing the zinc salt of DPTA and the Ammonium Salt of Etidronic Acid (Ethanehydroxybisphosphonate).

Liposomes containing both the ammonium salt of DPTA are prepared as described in Example 11 and the zinc salt of etridronic acid are prepared as described in Example 26. These liposomes have both an ammonium and zinc concentration gradient and are used to remote load either DFO as describe in Example 11 or deferasirox as described in Example 23. The resulting liposomes contain a three chelating agent combination and could be used to remove both plutonium and uranium from a contaminated individual. Other three chelating agent combinations that depend upon remote loading to load or more of the chelating agents could be prepared using the methods described in this invention.

### Example 28

Remote Loading of DFO into Liposomes Containing Multiple Cations to Remedy the Ability of DFO to Remove Essential Trace Metals when Administered to Patients.

As described in paragraph 8 current chelation therapies remove other trace metals including zinc and copper which may have adverse effects for the patients. As shown in Example 14, DFO can be remote loaded in liposomes containing ammonium sulfate and zinc sulfate. A mixture of ammonium sulfate, zinc sulfate and copper sulfate can also be encapsulated in the liposome at a ratio of the divalent metals that will compensate for the trace metals that can be removed by DFO when administered to patients. A suitable mole ratio of these cations is: ammonium/zinc/copper: 100/10/1.

### Example 29

Encapsulation of a Combination of Metal Acetates to Remote Load Deferasirox into Liposomes to Compensate for the Potential Removal of Trace Metals in a Patient by Deferasirox.

Liposomes composed of POPC/Cholesterol (3.0 mol/2.0 mol) containing either a mixture of 200 mM magnesium acetate, 20 mM zinc acetate and 2.0 mM copper acetate were prepared as described in Example 23. Addition of deferasirox dissolved in DMSO to the liposomes at a ratio near 240 g drug/mol phospholipid results in a final encapsulated drug ratio of >230 g drug/mol phospholipid (>96.0% efficiency) in the liposomes containing the mixture. The liposome remote loading capacity for deferasirox is dependent primarily upon the internal concentration of the magnesium acetate.

### Example 30

The Effect of Lipid Composition on In Vivo Stability of Actively Loaded DFO Containing Liposomes.

Liposomes containing egg PC/Chol (3 mol/2 mol, LDFO-1), egg PC/DOPG/Chol (2.7 mol/0.3 mol/2 mol, LDFO-2), and HSPC/Chol (3 mol/2 mol, LDFO-4) containing 500 mM ammonium sulfate were prepared as in Example 1. DFO was remote loaded as in Example 20.

After loading the liposomes were purified by dialysis versus HBS pH 6.5. The liposomes were sterilized by filtration through 0.2 um PES filters and analyzed for drug and lipid content. The liposomal DFO were administered to CF-1 female mice at 100 mg/kg (n=3) and at various times the animals were sacrificed and blood taken.

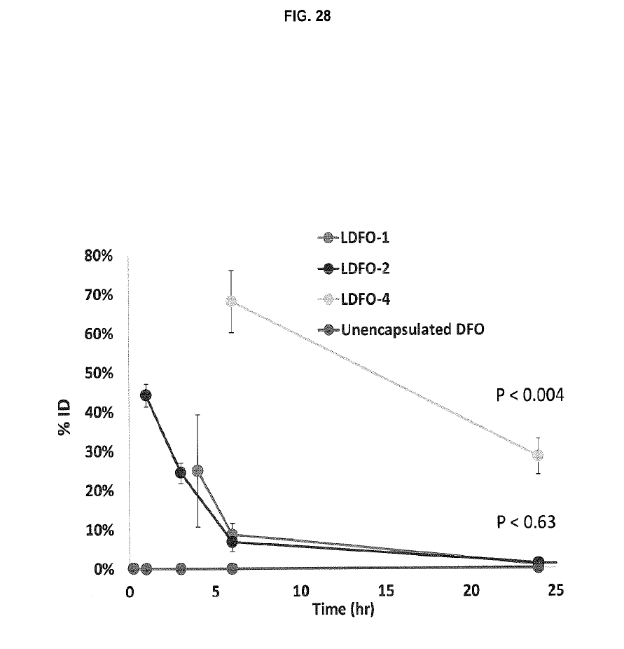

After processing to plasma, DFO was extracted from plasma by mixing with 6 volumes of methanol containing 0.1% trifluoroacetic acid in an eppendorf tube. The mixtures were vortexed twice for 10 seconds then placed at −80° C. for 1 h. The samples were thawed and centrifuged for 12,000 rpm for 10 min at 4° C. and the supernatant analyzed for DFO by HPLC as described herein. The pharmacokinetic plots of LDFO-1, -2 and 4 after IV administration are shown in FIG. 28. We observed that liposome encapsulation of DFO resulted in a very large increase in the amount of DFO in plasma compared to the non-encapsulated DFO when delivered at 100 mg/kg. LDFO-1 and LDFO-2 retained about 1% of the initial dose (ID) of DFO in the plasma at 24 h and LDFO-4 was the most stable with 30% ID retention. By comparison DFO was undetectable beyond 1 h after dosing with Desferal®.

The higher stability of LDFO-4 is attributable to the use of the more saturated lipid HSPC versus egg PC or DOPG used in LDFO-1 and -3. The highly saturated lipids appear to be more resistant to DFO leaking out of the liposome while in circulation.

### Example 31

The Effect of Metal on In Vivo Stability of Actively Loaded DFO Containing Liposomes.

Egg PC/Cholesterol liposomes (3 mol/2 mol) containing 500 mM ammonium sulfate were prepared as in Example 1. DFO was remote loaded as in Example 20. After loading the liposomes were purified by tangential flow filtration (TFF, Spectrum MidiKros 500 k MWCO PES cartridge) and exchanged into 50 mM Hepes buffered sucrose pH 6.5. The liposomes were analyzed for DFO and lipid content. The liposome solution was divided into five aliquots. One aliquot served as a control, no metals were added. Magnesium sulfate, zinc acetate, copper sulfate, barium acetate were added to each of the remaining 4 aliquots at a final concentration of 100 mM. The ionophore A23187 was used to shuttle metal cations inside the DFO loaded liposome. From a stock solution of A23187 (Sigma) (5 mg/ml in DMSO) aliquots were added to each of the metal salt/liposome containing solutions at 1.5 ug/mg lipid while vortexing. The liposome solutions were stirred overnight at 4° C.—then put on dialysis against 10% unbuffered sucrose and then exhaustively dialyzed against Hepes buffered sucrose pH 6.5 at 4° C.

The liposomes were sterilized by filtration through 0.2 um PES filters and analyzed for drug and lipid content. The liposomal DFO were administered to CF-1 female mice at 50 mg/kg (n=3) and at 6 h the animals were sacrificed and blood taken.

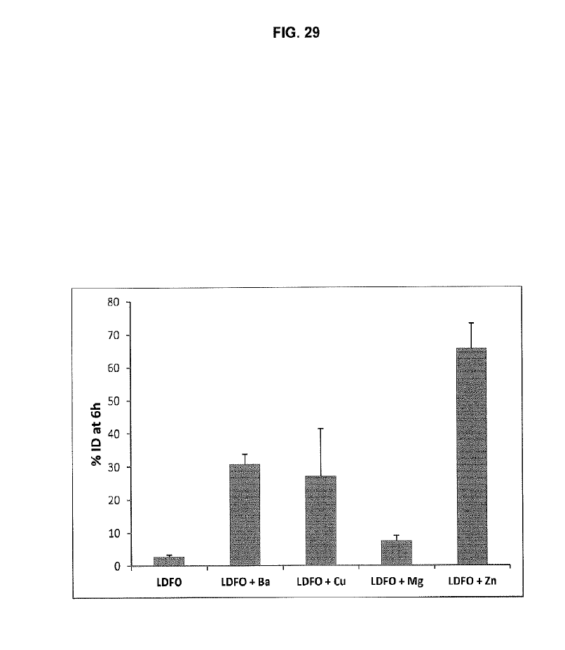

After processing to plasma, DFO was extracted from plasma by mixing with 6 volumes of methanol containing 0.1% trifluoroacetic acid in an eppendorf tube. The mixtures were vortexed twice for 10 seconds then placed at −80° C. for 1 h. The samples were thawed and centrifuged for 12,000 rpm for 10 min at 4° C. and the supernatant analyzed for DFO by HPLC as described herein. The results are shown below in FIG. 29.

The stability of liposomal DFO (LDFO) in vivo was dramatically dependent on the cation used. LDFO made using Egg PC/Chol have less than 3% of the chelator remaining in the blood at 6 h while LDFO with Zn added via the ionophore had over 65.5% remaining. Barium, copper and magnesium had 30.8, 27.1 and 7.5% remaining respectively. Therefore, liposomal DFO can be stabilized in vivo by adding metals into the interior of the liposome after the drug is loaded via an ammonium sulfate gradient.

### Example 32

Iron Excretion from Iron Overloaded Mouse Model Using LDFO Remote Loaded with Ascorbate Phosphate as the Trapping Agent.

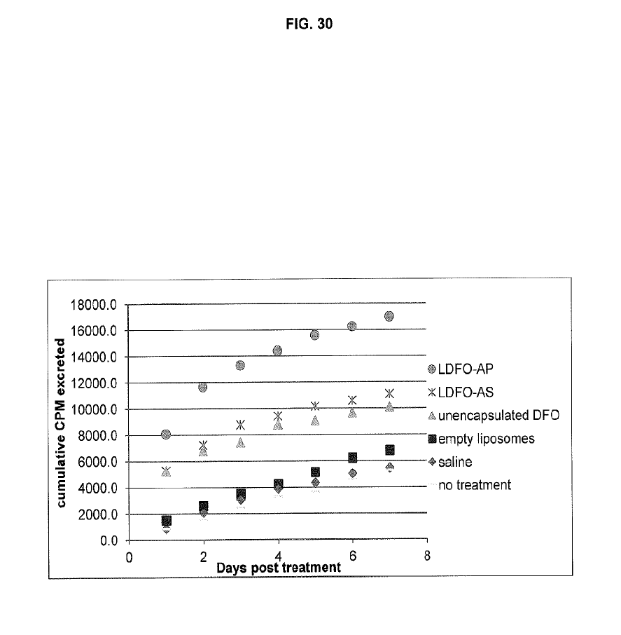

LDFO formulations were prepared as described in Example 1. Both formulations were composed of egg PC/cholesterol (3/0.5 (mol/mol)). One formulation was prepared using 250 mM ammonium sulfate (LDFO-AS) as the trapping agent and the second formulation was prepared using 250 mM ascorbate phosphate (LDFO-AP) as the trapping agent and all other components and DFO loading procedures of the formulations were identical. Mice were overloaded with iron-dextran for 4 weeks (2 mg/mouse IP once per week) and 59Fe-citrate dosed IP on week 5 at 10 μCi 59Fe chloride according to a protocol (described in Young et al. 1979). Fourteen days post-iron loading the mice (n=7) were treated with two prototypes formulations of LDFO and an unencapsulated DFO control all dosed at 100 mg/kg of chelator. The mice were housed in metabolic cages for 7 days for collection of urine. Urine radioactivity was measured via scintillation counting and the results shown in FIG. 30.

Enhanced ⁵⁹Fe removal was observed for both LDFO formulations tested when compared to unencapsulated DFO dosed at the same level (100 mg/kg chelator). LDFO-AS was slightly more effective than unencapsulated DFO but formulation LDFO-AP was approximately 1.6-fold better at removing iron via urinary excretion.

### Example 33

Iron Liver Removal from Iron Overloaded Mouse Model Using LDFO Remote Loaded with Ascorbate Phosphate as the Trapping Agent.

LDFO formulations were prepared as described in Example 1. Both formulations were composed of HSPC/cholesterol (3/0.5 (mol/mol)). One formulation was prepared using 250 mM ammonium sulfate (LDFO-AS) as the trapping agent and the second formulation was prepared using 250 mM ascorbate phosphate (LDFO-AP) as the trapping agent and all other components and DFO loading procedures of the formulations were identical. Mice were overloaded with a single IV injection of iron-dextran at 100 mg/kg Fe. Overloaded mice were treated with each formulation IV at 100 mg/kg chelator on day 7 and day 10. The mice were sacrificed after the second treatment and the livers were analyzed for iron content using a ferrozine assay (Riemer et al. 2004)

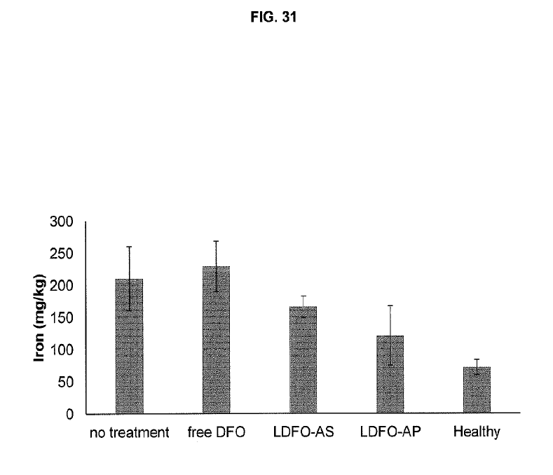

The mean liver iron content after treating with free DFO was 229 mg/kg and this values was 28% lower (p=0.18) after treating with LDFO-AS and 47% lower (p=0.009) after treating with LDFO-AP (FIG. 31).

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the claims appended hereto and their equivalents.

All publications, patents, and patent applications cited herein are hereby incorporated by reference in their entirety for all purposes.

### REFERENCES

Brittenham, G M, Iron-Chelating Therapy for transfusional Iron Overload, N Engl J Med 2011; 364:146-56. PMID: 21226580.
P. R. Cullis et al., Influence of pH gradients on the transbilayer transport of drugs, lipids, peptides and metal ions into large unilamellar vesicles. Biochimica et Biophysica Acta, 1997, 1331 187-211. PMID: 9325441.
S. De Virgiliis et al. Depletion of trace elements and acute ocular toxicity induced by desferrioxamine in patients with thalassemia, Arch. Of Disease in Childhood 63: 250-255, 1988.
D. Drummond et al. Pharmacokinetics and in vivo drug release rates in liposomal nanocarrier development. J Pharm Sci. 2008, 97(11):4696-740. PMID: 18351638.
Fleming R E, Ponka P., Iron overload in human disease. N Engl J Med. 2012 Jan. 26; 366(4):348-59. doi: 10.1056/NEJMra1004967. PMID: 22276824.
G. Gregoriadis Ed., Liposome Technology, Volume I, CRC Press, Boca Raton, Fla., (1984)
G. Gregoriadis, ed. Liposome Technology: Liposome Preparation and Related Techniques, 3rd Edition 2006.
Guilmette R A, Cerny E A, Rahman Y E. Pharmacokinetics of the iron chelating agent desferrioxamine as affected by liposome encapsulation: potential in treatment of chronic hemosiderosis. Life Sci. 22(4):313-2, 1978. PubMed PMID: 622008.
Ihnat P M, Vennerstrom J L, Robinson D H. Synthesis and solution properties of deferoxamine amides. J Pharm Sci. 2000 December; 89(12):1525-36.PMID: 11042600.
Lau E H, Cerny E A, Rahman Y E. Liposome-encapsulated desferrioxamine in experimental iron overload. Br J Haematol. 47(4):505-18, 1981. PubMed PMID: 7213574
Nick, H, Deferasirox (Exjade®, ICL670) Preclinical Overview, Seminars in Hematology 44:S12-S15, 2007.
Postma N S, Boerman O C, Oyen W J, Zuidema J, Storm G. Absorption and biodistribution of 111indium-labelled desferrioxamine (111In-DFO) after subcutaneous injection of 111In-DFO liposomes. J Control Release. 1999 Mar. 8; 58(1):51-60. PubMed PMID: 10021489.
Rahman Y E, Rosenthal M W, Cerny E A. Intracellular plutonium removal by liposome-encapsulated chelating agent. Science (Wash. D.C.) 180:300-302, 1973. PMID: 4633691.
Riemer J, Hoepken H H, Czerwinska H, Robinson S R, Dringen R. Colorimetric ferrozine-based assay for the quantitation of iron in cultured cells. Anal Biochem. 2004; 331(2):370-375. PubMed PMID: 15265744.
Young S P, Baker E, Huehns E R., Liposome entrapped desferrioxamine and iron transporting ionophores: a new approach to iron chelation therapy. Br J Haematol. 41(3):357-63, 1979. PubMed PMID: 4633691.
Weinberg E D, Miklossy J. Iron withholding: a defense against disease. J Alzheimers Dis. 2008 May; 13(4):451-63. PMID: 18487852.
D. Zucker et al., Liposome drugs' loading efficiency: a working model based on loading conditions and drug's physicochemical properties. J Control Release. 2009, 139: 73-80. PMID: 19508880.

What is claimed is:

1. A liposome comprising an encapsulated chelating agent, said liposome having a chelating agent:lipid ratio of greater than about 220 grams of said chelating agent per mole of lipid comprising said liposome, said liposome having a diameter from about 20 nm to about 300 nm.

2. The liposome according to claim 1, wherein said chelating agent is water soluble and selected from: ethylenediamine tetracetic acid (EDTA) also known as ethylenediamine tetraacetic acid (calcium disodium versante), diethylenetriaminepentaacetic acid (DTPA), deferoxamine, deferiprone, pyridoxal isonicotinoyl hydrazone, rhodotorulic acid, picolinic acid, nicotinic acid, neoaspergillic acid, methionine, lactic acid, N,N-ethylene bis[N-phosphonomethyl]glycine, tetraethylenepentaamine heptaacetic acid (TPHA), tri(2-aminoethyl)aminehexaacetic acid (TAAHA), triethylenetetraaminehexaacetic acid (TTHA), oxybis(ethylenenitrilo)tetraacetic acid (BAETA), trans-1,2-cyclohexaneediaminetetraacetic acid, salicyclic acid, tartaric acid, 2,3-dihydroxybenzoic acid, penicillamine, etidronic acid (1-hydroxyethan-1,1-diyl)bis(phosphonic acid), dimercaptosuccinic acid, dimercapto-propane sulfonate, and dimercaprol, desferrithiocin (DFT), polycarboxylates, hydroxamates, catecholates, hydroxypyridonates, teraphthalamides and analogues or derivatives of each water-soluble.

3. The liposome according to claim 1, wherein said chelating agent is sparingly water soluble and selected from: deferasirox, HBED (N,N′-bis(2-hydroxbenzyl)ethylenediamine-N—N-diacetic acid), HBPD (N,N′-Bis(2-hydroxybenyzyl)propylene-1,3-diamine-N,N′-diacetic acid.

4. The formulation according to claim 1, wherein said liposome is loaded with water soluble chelating agent in response to an electrochemical gradient with the aid of a membrane permeability enhancer such as methanol, ethanol, propanol, butanol or t-butanol or other alcohols.

5. The formulation according to claim 1, wherein said liposome is loaded with a sparingly soluble chelating agent with the aid of a precipitate enabling solvent, wherein the precipitate enabling solvent is a polar aprotic solvent, said polar aprotic solvent essentially completely dissolving the chelating agent, said polar aprotic solvent selected from acetone, acetonitrile, N,N′ dimethyllformamide, dimethylacetamide, dioxane, dimethylsulfoxide (DMSO), ethylacetate, hexamethylphosphorotriamide, glyme (dimethylethoxyethane), N-methyl-2-pyrrolidone, sulfolane, tetrahydrofuran, said liposome prepared by a method comprising: forming a chelating agent solution by dissolving chelating agent in said polar aprotic solvent and combining said chelating agent solution with an aqueous medium comprising said liposome such that the chelating agent becomes insoluble and precipitates prior to loading inside liposome.

6. The formulation according to claim 1, wherein said liposome is loaded with a chelating agent in response to an electrochemical gradient at a temperature from 70° C. to 120° C.

7. The formulation according to claim 1, wherein said liposome containing a loaded chelating agent in response to an electrochemical gradient is stabilized by placing it a −10° C. to 10° C. for at least one hour after remote loading.

8. The formulation according to claim 1, wherein said liposome containing a loaded chelating agent also contains a mixture of cations at the following mole fraction: ammonium (0-1), calcium (0-1), magnesium (0-1), zinc (0-0.3), copper (0-0.03), sodium (0-0.3) so that the combination mole fraction of ammonium+calcium+magnesium+zinc+copper+sodium totals to 1.00.

9. The liposome of claim 1, wherein said chelating agent is deferoxamine.

10. The liposome of claim 1, wherein said chelating agent is deferasirox.

11. A pharmaceutical formulation comprising a liposome according to claim 1 in a pharmaceutically acceptable vehicle.

12. The formulation according to claim 1, wherein said liposome comprises a chelating agent encapsulated within said liposome, said formulation manufactured by a method comprising:
contacting an aqueous suspension of said liposome with a solution of said chelating agent under conditions appropriate to encapsulate said water-soluble chelating agent in said liposome, wherein
said liposome has an internal aqueous environment encapsulated by a lipid membrane and said aqueous suspension of said liposome comprises a proton and/or ion gradient across said membrane, and wherein
said conditions are appropriate for said sparingly water-soluble chelating agent to traverse said membrane and concentrate in said internal aqueous environment, thereby forming said pharmaceutical formulation.

13. The liposome of claim 1, where said liposome is selected from multilamellar vesicles (MLV), large unilamellar vesicles (LUV) and small unilamellar vesicles (SUV), oligolamellar vesicles (OLV), paucilamellar vesicles (PLV) or reverse phase evaporation vesicles (REV).

14. The liposome of claim 1, wherein the liposome is prepared from one or more lipids selected from egg phosphatidylcholine (EPC), egg phosphatidylglycerol (EPG), dipalmitoylphosphatidylcholine (DPPC), dioleolylphosphatidylcholine (DOPC), palmitoyloleoylphosphatidylcholine (POPC), sphingomyelin (SM), cholesterol (Chol), cholesterol sulfate and its salts (CS), cholesterol hemisuccinate and its salts (Chems), cholesterol phosphate and its salts (CP), cholesterylphosphorylcholine, dimyristoylphosphatidylglycerol (DMPG), dimyristoylphosphatidylglycerol (DMPG), dimyristoylphosphatidylcholine (DMPC), distearoylphosphatidylcholine (DSPC), soy phosphatidylcholine, hydrogenated soy phosphatidylcholine (HSPC), distearoylphosphatidylglycerol (DSPG), sterol modified lipids (SML), inverse-phosphocholine lipids, cationic lipids and zwitterlipids.

15. The liposome of claim 1, wherein the encapsulation efficiency of the initial amount of chelating agent that is incorporated into the final liposome ranges from about 30% to about 100% of the initial amount of chelating agent.

16. The liposome of claim 12, wherein said ion gradient is caused by a difference in concentrations across said membrane of a member selected from an amine salt of a carboxylate, a sulfate, a phosphate, a phosphonate, or an acetate.

17. The liposome of claim 16, wherein said amine salt is selected from a monovalent carboxylate, a multivalent carboxylate, a sulfate, and a phosphate.

18. The liposome of claim 12, wherein said ion gradient is caused by a difference in concentrations across said membrane of a member selected from an acetate salt of a cation.

19. The liposome of claim 18, wherein the cation in said acetate salt is selected from sodium, potassium, barium, calcium, cobalt, copper, magnesium, manganese, nickel, zinc, primary, secondary, tertiary and quaternary ammonium species.

20. A formulation comprising a liposome of claim 1, wherein said formulation is lyophilized.

21. A formulation comprising a liposome of claim 1, wherein said chelating agent is present in said formulation as a unit dosage format.

22. A method of treatment comprising decorporating a metal ion from a subject in need of said treatment, said method comprising administering to said patient a therapeutically effective amount of said formulation of claim 1.

23. A formulation comprising a liposome of claim 1, wherein two or more chelating agents are present in said formulation.

24. A kit comprising: a first container comprising a chelating agent; a second container comprising a liposome suspension with an ion gradient such that the ion concentration is higher inside of said liposome than outside said liposome; a third container of a buffer; and directions for combining contents of said first, second and third containers to form a liposome of claim 1.

25. The kit according to claim 21, wherein a member selected from said liposome, chelating agent and a combination thereof are in dry or lyophilized form.
